SEQUENCE LISTING (13 sequences)

SEQ ID NO: 1 - DNA, 2949 bp, Caenorhabditis elegans
atggcgcctgccgtcttacaaaaacccggtgttatcaaggatccatcgattgctgcattgttcagtaataaggatccagagcagagatatcaagatttaagagaaattggacatggatcttttggagctgtctattttgcatatgacaaaaaaaatgagcagactgttgcgattaaaaagatgaattttagtggaaaacaggctgtcgaaaaatggaatgatattcttaaagaagtgtcttttctgaatacagttgttcatccacatattgtcgactacaaggcttgttttcttaaggacactacatgttggcttgtgatggagtactgtattggctctgcagccgatatagtggatgtcttgcgaaaaggaatgcgagaagtcgaaatcgctgcgatttgctctcaaactttggatgctcttcgatatcttcactctctgaagcgaatacatcgagatattaaagctggaaatattctgctatctgatcatgctattgttaaactagctgatttcggatccgcatccctggtagatccggctcaaactttcatcggaacgccgtttttcatggccccagaggtaattctggcaatggatgagggtcactacacggatcgtgcagatatttggtcattgggtatcacgtgtatagagctggccgaacgtcgtccaccattgttcagtatgaatgcaatgtctgccctctaccatattgctcaaaatgatcctccaactctttctccaattgacactagcgaacaaccggaatggtcgctggaattcgttcaatttatagacaaatgtcttcgaaaaccagcagaagagcgaatgtcagctgaagaatgctttcgacatccattcattcaacggtctcgcccatcagacacaattcaggaactcattcagagaacgaaaaatatggtattagagttggataattttcaatacaaaaagatgagaaaactcatgtatttggatgaaacagaaggaaaagaaggaagtgaaggaaatggagcatctgatgatttagattttcatggaaatgaagctaattcaattggaagagcaggagattctgcgtcatctcgaagtgcttctcttacttctttccgatcaatgcagagtagtggaggagctggtcttttagtgtccaccaatacgacgggtgctatggataatgtgcatggatcctctggatacggtaatggaagtagttcgacgacgagctccgcacgccgccgtcctccaattccttcgcaaatgctctcttctacatcaacgtctggtgttggaactatgccgagtcatggatcagttggagcatcgattacggcgatcgcagtcaatccaacaccgtctccttcagaacctatcccaacatcacaaccaacatcgaaatcagaatcatcttctatactcgaaactgcacacgatgatcctttggacacgtcgatacgtgctccagtgaaagacttgcatatgccgcatcgagcagtcaaggaacgaatagccacgttgcaaaatcacaaattcgcgacgcttcgttcccagagaataatcaatcaggaacaagaagaatatacgaaagagaacaatatgtatgagcaaatgagcaagtacaagcatctacgacaagcacatcacaaagagctccaacaatttgaagaacgatgtgcattagatagagagcaactgcgtgtgaaaatggatcgagaactcgaacaattgacaacgacatactcgaaagaaaagatgagagtgaggtgttcacagaataatgaactagacaaacggaaaaaagatatcgaagatggggagaaaaagatgaaaaagacgaaaaatagtcaaaatcagcagcagatgaaactgtattcagcgcaacaattgaaagaatacaagtataacaaggaggcacagaaaacacgattacgaagtctgaacatgcctcgaagtacttatgagaacgcaatgaaagaagtgaaagccgatctgaatcgagtgaaagatgcacgggaaaatgattttgacgagaagcttcgtgcagaacttgaagatgaaattgtaaggtatcgcaggcaacaactcagtaatcttcatcaattggaagaacaattggatgatgaagacgtaaacgtgcaagaacgccaaatggacacgcgtcacggattactgtcaaagcagcatgaaatgacgcgcgatttggaaatacagcatctcaacgagcttcacgcgatgaaaaaacgacatttggagacacaacacgaggcggaatcggcaagtcaaaatgagtacacacagaggcaacaggatgaattgagaaaaaagcatgcgatgcagtcaagacaacagccaagagatttaaagatccaagaagcacaaattcgaaaacaataccgacaagttgtgaagactcagactcgccaatttaagctctaccttacacaaatggtgcaagtagttccaaaagatgaacaaaaagagctcacgtctcgactaaaacaggatcaaatgcaaaaagtcgcacttcttgcttcacaatacgaaagtcaaatcaaaaaaatggttcaggataagacagtgaagctcgagtcgtggcaagaagatgaacaacgggttcttagtgagaagttggagaaagaattggaagaattgattgcttatcagaagaagacgagagccacattagaagagcagattaaaaaggaacgtacggcactcgaagaacgaattggcacacgacgtgcaatgcttgaacagaagattattgaagaacgcgaacaaatgggagaaatgcgtcgactaaagaaggagcaaatccgtgatcgacacagtcaagaacgccatcgtctcgagaatcatttcgtacggacgggctcgacgagcagaagttctggtgggatcgctcctggtgttgggaattcaagcagtattcagatggctatgtag

SEQ ID NO: 2 - protein, 982 aa, Caenorhabditis elegans
MAPAVLQKPGVIKDPSIAALFSNKDPEQRYQDLREIGHGSFGAVYFAYDKKNEQTVAIKKMNFSGKQAVEKWNDILKEVSFLNTVVHPHIVDYKACFLKDTTCWLVMEYCIGSAADIVDVLRKGMREVEIAAICSQTLDALRYLHSLKRIHRDIKAGNILLSDHAIVKLADFGSASLVDPAQTFIGTPFFMAPEVILAMDEGHYTDRADIWSLGITCIELAERRPPLFSMNAMSALYHIAQNDPPTLSPIDTSEQPEWSLEFVQFIDKCLRKPAEERMSAEECFRHPFIQRSRPSDTIQELIQRTKNMVLELDNFQYKKMRKLMYLDETEGKEGSEGNGASDDLDFHGNEANSIGRAGDSASSRSASLTSFRSMQSSGGAGLLVSTNTTGAMDNVHGSSGYGNGSSSTTSSARRRPPIPSQMLSSTSTSGVGTMPSHGSVGASITAIAVNPTPSPSEPIPTSQPTSKSESSSILETAHDDPLDTSIRAPVKDLHMPHRAVKERIATLQNHKFATLRSQRIINQEQEEYTKENNMYEQMSKYKHLRQAHHKELQQFEERCALDREQLRVKMDRELEQLTTTYSKEKMRVRCSQNNELDKRKKDIEDGEKKMKKTKNSQNQQQMKLYSAQQLKEYKYNKEAQKTRLRSLNMPRSTYENAMKEVKADLNRVKDARENDFDEKLRAELEDEIVRYRRQQLSNLHQLEEQLDDEDVNVQERQMDTRHGLLSKQHEMTRDLEIQHLNELHAMKKRHLETQHEAESASQNEYTQRQQDELRKKHAMQSRQQPRDLKIQEAQIRKQYRQVVKTQTRQFKLYLTQMVQVVPKDEQKELTSRLKQDQMQKVALLASQYESQIKKMVQDKTVKLESWQEDEQRVLSEKLEKELEELIAYQKKTRATLEEQIKKERTALEERIGTRRAMLEQKIIEEREQMGEMRRLKKEQIRDRHSQERHRLENHFVRTGSTSRSSGGIAPGVGNSSSIQMAM

SEQ ID NO: 3 - DNA, 4188 bp, Homo sapiens
  ref <308>: DATABASE ACCESSION NUMBER: genbank NM_016281
  ref <309>: DATABASE ENTRY DATE: 2002-10-04
  ref <313>: RELEVANT RESIDUES: (1)..(4188)
gccggggaacaagccacaggagagcgactcaggaacaagtgtgggagaggaagcggcggcggcggcgccgggcccgggggtggtgacagcaggtctgaggttgcatcataaatacaaaggactgaagttataaaagagaaaagagaagtttgctgctaaaatgaatctgagcaatatggaatattttgtgccacacacaaaaaggtactgaagatttaccccccaaaaaaaattgtcaatgagaaataaagctaactgatatcaaaaagcagagcctgctctactggccatcatgcgtaaaggggtgctgaaggacccagagattgccgatctattctacaaagatgatcctgaggaactttttattggtttgcatgaaattggacatggaagttttggagcagtttattttgctacaaatgctcacaccaatgaggtggtggcaattaagaagatgtcctatagtgggaagcagacccatgagaaatggcaagatattcttaaggaagttaaatttttacgacaattgaagcatcctaatactattgagtacaaaggctgttacttgaaagaacacactgcttggttggtgatggaatattgcttaggctcagcctctgatttattagaagttcataaaaaaccacttcaggaagtggagatcgctgccattactcatggagccttgcatggactagcctacctacattctcatgcattgattcatagggatattaaagcaggaaatattcttctaacagagccaggtcaggtaaaactagctgattttggatctgcttcaatggcttctcctgccaactccttcgtgggcacaccttactggatggctccagaggtgatcttagctatggatgaaggacagtatgatgggaaagttgatatttggtcacttggcatcacttgtattgaattggcggaacggaagccgccccttttcaacatgaatgcaatgagtgccttatatcacattgcccagaatgactccccaacgttacagtctaatgaatggacagactcctttaggagatttgttgattactgcttgcagaaaatacctcaggaaaggccaacatcagcagaactattaaggcatgactttgttcgacgagaccggccactacgtgtcctcattgacctcatacagaggacaaaagatgcagttcgtgagctagataacctacagtaccgaaaaatgaaaaaaatacttttccaagagacacggaatggacccttgaatgagtcacaggaggatgaggaagacagtgaacatggaaccagcctgaacagggaaatggacagcctgggcagcaaccattccattccaagcatgtccgtgagcacaggcagccagagcagcagtgtgaacagcatgcaggaagtcatggacgagagcagttccgaacttgtcatgatgcacgatgacgaaagcacaatcaattccagctcctccgtcgtgcataagaaagatcatgtattcataagggatgaggcgggccacggcgatcccaggcctgagccgcggcctacccagtcagttcagagccaggccctccactaccggaacagagagcgctttgccacgatcaaatcagcatctttggttacacgacagatccatgagcatgagcaggagaacgagttgcgggaacagatgtcaggttataagcggatgcggcgccagcaccagaagcagctgatcgccctggagaacaagctgaaggctgagatggacgagcaccgcctcaagctacagaaggaggtggagacgcatgccaacaactcgtccatcgagctggagaagctggccaagaagcaagtggctatcatagaaaaggaggcaaaggtagctgcagcagatgagaagaagttccagcaacagatcttggcccagcagaagaaagatttgacaactttcttagaaagtcagaagaagcagtataagatttgtaaggaaaaaataaaagaggaaatgaatgaggaccatagcacacccaagaaagagaagcaagagcggatctccaaacataaagagaacttgcagcacacacaggctgaagaggaagcccaccttctcactcaacagagactgtactacgacaaaaattgtcgtttcttcaagcggaaaataatgatcaagcggcacgaggtggagcagcagaacattcgggaggaactaaataaaaagaggacccagaaggagatggagcatgccatgctaatccggcacgacgagtccacccgagagctagagtacaggcagctgcacacgttacagaagctacgcatggatctgatccgtttacagcaccagacggaactggaaaaccagctggagtacaataagaggcgagaaagagaactgcacagaaagcatgtcatggaacttcggcaacagccaaaaaacttaaaggccatggaaatgcaaattaaaaaacagtttcaggacacttgcaaagtacagaccaaacagtataaagcactcaagaatcaccagttggaagttactccaaagaatgagcacaaaacaatcttaaagacactgaaagatgagcagacaagaaaacttgccattttggcagagcagtatgaacagagtataaatgaaatgatggcctctcaagcgttacggctagatgaggctcaagaagcagaatgccaggccttgaggctacagctccagcaggaaatggagctgctcaacgcctaccagagcaaaatcaagatgcaaacagaggcacaacatgaacgtgagctccagaagctagagcagagagtgtctctgcgcagagcacaccttgagcagaagattgaagaggagctggctgcccttcagaaggaacgcagcgagagaataaagaacctattggaaaggcaagagcgagagattgaaacttttgacatggagagcctcagaatgggatttgggaatttggttacattagattttcctaaggaggactacagatgagattaaattttttgccatttacaaaaaaaaaaaaaaaaagaaaacagaaaaaaattcagaccctgcaaaaccacattccccattttaacgggcgttgctctcactctctctctctcttactcttactgacatcgtgtcggactagtgcctgtttattcttactccatcaggggcccccttcctccccccgtgtcaactttcagtgctggccaaaacctggccgtctcttctattcacagtacacgtcacagtattgatgtgattcaaaatgtttcagtgaaaactttggagacagttttaacaaaaccaataaaccaacaacaaaaaaagtggatgtatattgctttaagcaatcactcattaccaccaatctgtgaaagtaaagcaaaaaataataataataaatgccaagggggagagagacacaatatccgcagccttacaccttaactagctgctgcattattttattttattttatttttttggtatttattcatcaggaataaaaaaaacaaagttttattaaagattgaaaatttgatacattttacagaaactaattgtgatgtacatatcagtggtgacatattattacttttttggggacgggggtgggtggggtgaagagatcttgtgattttagactgctgcagagttaacttgtctcagcatatctgatgtatcataatcatttctgctgtgcagaggagggatacacttaggggctcacagatcccagtagcacaattgggctttggcaaatgggtattttgtgtatagaggaatttaaggagaggtattacttattttcatattgtattttaactgtttctcggatcaaattttttaacttcttcttcgtgttcttccccacctccttccttttccagttcagtatttggagttcaacactgtctctcaatcagatcatctggatctttttctttatctcccttccccttcctaagtcccatttcttggtcataaatattgcattattcacactttcaaactgtgtattttcttacaataaaaaatgatgaaaaaaaaaaaggctttacttcttttgcatgcactttaaaaacaaaacaaaacatttttcaggttccaaggaagagcatgataactgtcagagcttttaattatatttgtaaataaaagtgttcatcacaaaaaaaaaaaaaaaaaaaaaaaaa

SEQ ID NO: 4 - protein, 898 aa, Homo sapiens
  ref <308>: DATABASE ACCESSION NUMBER: Genbank NP_057365.2
  ref <309>: DATABASE ENTRY DATE: 2002-10-01
  ref <313>: RELEVANT RESIDUES: (1)..(898)
MRKGVLKDPEIADLFYKDDPEELFIGLHEIGHGSFGAVYFATNAHTNEVVAIKKMSYSGKQTHEKWQDILKEVKFLRQLKHPNTIEYKGCYLKEHTAWLVMEYCLGSASDLLEVHKKPLQEVEIAAITHGALHGLAYLHSHALIHRDIKAGNILLTEPGQVKLADFGSASMASPANSFVGTPYWMAPEVILAMDEGQYDGKVDIWSLGITCIELAERKPPLFNMNAMSALYHIAQNDSPTLQSNEWTDSFRRFVDYCLQKIPQERPTSAELLRHDFVRRDRPLRVLIDLIQRTKDAVRELDNLQYRKMKKILFQETRNGPLNESQEDEEDSEHGTSLNREMDSLGSNHSIPSMSVSTGSQSSSVNSMQEVMDESSSELVMMHDDESTINSSSSVVHKKDHVFIRDEAGHGDPRPEPRPTQSVQSQALHYRNRERFATIKSASLVTRQIHEHEQENELREQMSGYKRMRRQHQKQLIALENKLKAEMDEHRLKLQKEVETHANNSSIELEKLAKKQVAIIEKEAKVAAADEKKFQQQILAQQKKDLTTFLESQKKQYKICKEKIKEEMNEDHSTPKKEKQERISKHKENLQHTQAEEEAHLLTQQRLYYDKNCRFFKRKIMIKRHEVEQQNIREELNKKRTQKEMEHAMLIRHDESTRELEYRQLHTLQKLRMDLIRLQHQTELENQLEYNKRRERELHRKHVMELRQQPKNLKAMEMQIKKQFQDTCKVQTKQYKALKNHQLEVTPKNEHKTILKTLKDEQTRKLAILAEQYEQSINEMMASQALRLDEAQEAECQALRLQLQQEMELLNAYQSKIKMQTEAQHERELQKLEQRVSLRRAHLEQKIEEELAALQKERSERIKNLLERQEREIETFDMESLRMGFGNLVTLDFPKEDYR

SEQ ID NO: 5 - DNA, 4971 bp, Homo sapiens
  ref <308>: DATABASE ACCESSION NUMBER: Genbank NM_016151.1
  ref <309>: DATABASE ENTRY DATE: 2002-11-05
  ref <313>: RELEVANT RESIDUES: (1)..(4971)
aattcggcacgagctgagacggagaagaggagaggcagagagggcgcggggaccgtcagcagcaccttagctacaatcgttcagctattctcggaagagagaagggagagggaggaggccggggcgggagtgggggctgtcaccctcggaccccggcgtgagaggggccgtgcggccggacgtcctcggggtgggcccccagtcggtggccgaagacctacagctcaggcccctgggtcccaaatttccaggctttgcccctcctcctttctcagatacccgggtaacagtcctcatagtccagatatccgggactcgggtcccaacctctctaaacctgggtctctgtttcatagattttcaaatatcaggttcaggcccctgcgtgcaccagtatccggggttcattccccgggcgtttcaaatatcggattcagtctccatcccgttcagatattcggggttcagaccccacaatcagaaatccggaattcggcagctgtcgccctcgacgagggggaggactggaccgcgaggtcagattaggttgtcaccccctcccctccaggggaggcttcccgggcccgcccctcaggaagggcgaaagccgaggaagaggtggcaaggggaaaggtctccttgcccctctccctgcttggcagagccgctggaggaccccaggcggaagcggaggcgctggggcaccatagtgacccctaccaggccaggccccactctcagggcccccaggggccaccatgccagctgggggccgggccgggagcctgaaggacccagatgtggctgagctcttcttcaaggatgacccagaaaagctcttctctgacctccgggaaattggccatggcagctttggagccgtatactttgcccgggatgtccggaatagtgaggtggtggccatcaagaagatgtcctacagtgggaagcagtccaatgagaaatggcaagacatcatcaaggaggtgcggttcttacagaagctccggcatcccaacaccattcagtaccggggctgttacctgagggagcacacggcttggctggtaatggagtattgcctgggctcagcttctgaccttctagaagtgcacaagaaaccccttcaggaggtagagatcgcagctgtgacccacggggcgcttcagggcctggcatatctgcactcccacaacatgatccatagggatgtgaaggctggaaacatcctgctgtcagagccagggttagtgaagctaggggactttggttctgcgtccatcatggcacctgccaactccttcgtgggcaccccatactggatggcacccgaggtgatcctggccatggatgaggggcagtacgatggcaaagtggacgtctggtccttggggataacctgcatcgagctggctgaacggaaaccaccgctctttaacatgaatgcgatgagtgccttataccacattgcacagaacgaatcccccgtgctccagtcaggacactggtctgagtacttccggaattttgtcgactcctgtcttcagaaaatccctcaagacagaccaacctcagaggttctcctgaagcaccgctttgtgctccgggagcggccacccacagtcatcatggacctgatccagaggaccaaggatgccgtgcgggagctggacaacctgcagtaccgcaagatgaagaagatcctgttccaagaggcacccaacggccctggtgccgaggccccagaggaggaagaggaggccgagccctacatgcaccgggccgggactctgaccagcctcgagagtagccactcagtgcccagcatgtccatcagcgcctccagccagagcagctccgtcaacagcctagcagatgcctcagacaacgaggaagaggaggaggaggaggaggaagaggaggaggaggaagaaggccctgaagcccgggagatggccatgatgcaggagggggagcacacagtcacctctcacagctccattatccaccggctgccgggctctgacaacctatatgatgacccctaccagccagagataacccccagccctctccagccgcctgcagccccagctcccacttccaccacctcttccgcccgccgccgggcctactgccgtaaccgagaccactttgccaccatccgaaccgcctccctggtcagccgtcagatccaggagcatgagcaggactctgcgctgcgggagcagctgagcggctataagcggatgcgacgacagcaccagaagcagctgctggccctggagtcacggctgaggggtgaacgggaggagcacagtgcacggctgcagcgggagcttgaggcgcagcgggctggctttggggcagaggcagaaaagctggcccggcggcaccaggccataggtgagaaggaggcacgagctgcccaggccgaggagcggaagttccagcagcacatccttgggcagcagaagaaggagctggctgccctgctggaggcacagaagcggacctacaaacttcgcaaggaacagctgaaggaggagctccaggagaaccccagcactcccaagcgggagaaggccgagtggctgctgcggcagaaggagcagctccagcagtgccaggcggaggaggaagcagggctgctgcggcggcagcgccagtactttgagctgcagtgtcgccagtacaagcgcaagatgttgctggctcggcacagcctggaccaggacctgctgcgggaggacctgaacaagaagcagacccagaaggacttggagtgtgcactgctgcttcggcagcacgaggccacgcgggagctggagctgcggcagctccaggccgtgcagcgcacgcgggctgagctcacccgcctgcagcaccagacggagctgggcaaccagctggagtacaacaagcggcgtgagcaagagttgcggcagaagcatgcggcccaggttcgccagcagcccaagagcctcaaagtacgtgcaggccagcgccccccgggccttccactccccattcctggggctctgggcccacccaacacaggcacccctatagaacagcagccctgctcacctggccaggaggcagtcctggaccaaagaatgcttggcgaggaggaggaagcagttggagagagaaggattctgggaaaggaaggggccactttggagcccaagcagcagaggattctgggggaagaatcaggagcccctagtcccagtccacaaaaacatgggagcctggttgatgaggaagtttggggtctgcctgaggagatagaggagcttagggtgccctcccttgtaccccaggagaggagcattgttggccaggaggaggctgggacgtggagcttgtgggggaaggaggatgagagtcttctggatgaggagtttgagcttggctgggtccagggcccagcactgactcccgtccctgaggaggaggaagaagaggaagagggggctccgattgggacccctagggatcctggagatggttgtccttcccccgacatccctcctgaaccccctccaacacacctgaggccctgccctgccagccagctccctggactcctgtcccatggcctcctggccggcctctcctttgcagtggggtcctcctctggcctcctgcccctcctgctgctgctgctgcttccattgctggcagcccagggtgggggtggcctgcaggcagcgctgctggcccttgaggtggggctggtgggtctgggggcctcctacctgctcctttgtacagccctgcacctgccctccagtcttttcctactcctggcccagggtaccgcactgggggccgtcctgggcctgagctggcgccgaggcctcatgggtgttcccctgggccttggagctgcctggctcttagcttggccaggcctagctctacctctggtggctatggcagcggggggcagatgggtgcggcagcagggcccccgggtgcgccggggcatatctcgactctggttgcgggttctgctgcgcctgtcacccatggccttccgggccctgcagggctgtggggctgtgggggaccggggtctgtttgcactgtaccccaaaaccaacaaggatggcttccgcagccgcctgcccgtccctgggccccggcggcgtaatccccgcaccacccaacacccattagctctgttggcaagggtctgggtcctgtgcaagggctggaactggcgtctggcacgggccagccagggtttagcatcccacttgcccccgtgggccatccacacactggccagctggggcctgcttcggggtgaacggcccacccgaatcccccggctactaccacgcagccagcgccagctagggccccctgcctcccaccagccactgccagggactctagccgggcggaggtcacgcacccgccagtcccgggccctgcccccctggaggtagctgactccagcccttccagcccaaatctagagcattgagcactttatctcccacgactcagtgaagtttctccagtccctagtcctctcttttcacccaccttcctcagtttgctcacttaccccaggcccagcccttcggacctctagacaggcagcctcctcagctgtggagtccagcagtcactctgtgttctcctggcgctcctcccctaagttattgctgttcgcccgctgtgtgtgctcatcctcaccctcattgactcaggcctggggccaggggtggtggagggtgggaagagtcatgttttttttctcctctttgattttgtttttctgtctcccttccaacctgtccccttccccccaccaaaaaaagaaaaagacaaacacaaataaaatatctgagcggaactgtgaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - protein, 1235 aa, Homo sapiens
  ref <308>: DATABASE ACCESSION NUMBER: Genbank NP_057235.1
  ref <309>: DATABASE ENTRY DATE: 2002-11-05
  ref <313>: RELEVANT RESIDUES: (1)..(1235)
MPAGGRAGSLKDPDVAELFFKDDPEKLFSDLREIGHGSFGAVYFARDVRNSEVVAIKKMSYSGKQSNEKWQDIIKEVRFLQKLRHPNTIQYRGCYLREHTAWLVMEYCLGSASDLLEVHKKPLQEVEIAAVTHGALQGLAYLHSHNMIHRDVKAGNILLSEPGLVKLGDFGSASIMAPANSFVGTPYWMAPEVILAMDEGQYDGKVDVWSLGITCIELAERKPPLFNMNAMSALYHIAQNESPVLQSGHWSEYFRNFVDSCLQKIPQDRPTSEVLLKHRFVLRERPPTVIMDLIQRTKDAVRELDNLQYRKMKKILFQEAPNGPGAEAPEEEEEAEPYMHRAGTLTSLESSHSVPSMSISASSQSSSVNSLADASDNEEEEEEEEEEEEEEEGPEAREMAMMQEGEHTVTSHSSIIHRLPGSDNLYDDPYQPEITPSPLQPPAAPAPTSTTSSARRRAYCRNRDHFATIRTASLVSRQIQEHEQDSALREQLSGYKRMRRQHQKQLLALESRLRGEREEHSARLQRELEAQRAGFGAEAEKLARRHQAIGEKEARAAQAEERKFQQHILGQQKKELAALLEAQKRTYKLRKEQLKEELQENPSTPKREKAEWLLRQKEQLQQCQAEEEAGLLRRQRQYFELQCRQYKRKMLLARHSLDQDLLREDLNKKQTQKDLECALLLRQHEATRELELRQLQAVQRTRAELTRLQHQTELGNQLEYNKRREQELRQKHAAQVRQQPKSLKVRAGQRPPGLPLPIPGALGPPNTGTPIEQQPCSPGQEAVLDQRMLGEEEEAVGERRILGKEGATLEPKQQRILGEESGAPSPSPQKHGSLVDEEVWGLPEEIEELRVPSLVPQERSIVGQEEAGTWSLWGKEDESLLDEEFELGWVQGPALTPVPEEEEEEEEGAPIGTPRDPGDGCPSPDIPPEPPPTHLRPCPASQLPGLLSHGLLAGLSFAVGSSSGLLPLLLLLLLPLLAAQGGGGLQAALLALEVGLVGLGASYLLLCTALHLPSSLFLLLAQGTALGAVLGLSWRRGLMGVPLGLGAAWLLAWPGLALPLVAMAAGGRWVRQQGPRVRRGISRLWLRVLLRLSPMAFRALQGCGAVGDRGLFALYPKTNKDGFRSRLPVPGPRRRNPRTTQHPLALLARVWVLCKGWNWRLARASQGLASHLPPWAIHTLASWGLLRGERPTRIPRLLPRSQRQLGPPASHQPLPGTLAGRRSRTRQSRALPPWR

SEQ ID NO: 7 - DNA, 4242 bp, Homo sapiens
  ref <308>: DATABASE ACCESSION NUMBER: Genbank NM_004783
  ref <309>: DATABASE ENTRY DATE: 2000-11-01
  ref <313>: RELEVANT RESIDUES: (1)..(4242)
agaatttcaaatatcaggttcaggcccctgcgtgcaccagtatccggggttcattccccgggcgttcaaatatcggattcagtctccatcccgttcagatattcggggttcagaccccacaatcagaaatccggaattcggcagctgtcgccctcgacgagggggaggactggaccgcgaggtcagattaggttgtcaccccctcccctccaggggaggcttcccgggcccgcccctcaggaagggcgaaagccgaggaagaggtggcaaggggaaaggtctccttgcccctctccctgcttggcagagccgctggaggaccccaggcggaagcggaggcgctggggcaccatagtgacccctaccaggccaggccccactctcagggcccccaggggccaccatgccagctgggggccgggccgggagcctgaaggacccagatgtggctgagctcttcttcaaggatgacccagaaaagctcttctctgacctccgggaaattggccatggcagctttggagccgtatactttgcccgggatgtccggaatagtgaggtggtggccatcaagaagatgtcctacagtgggaagcagtccaatgagaaatggcaagacatcatcaaggaggtgcggttcttacagaagctccggcatcccaacaccattcagtaccggggctgttacctgagggagcacacggcttggctggtaatggagtattgcctgggctcagcttctgaccttctagaagtgcacaagaaaccccttcaggaggtagagatcgcagctgtgacccacggggcgcttcagggcctggcatatctgcactcccacaacatgatccatagggatgtgaaggctggaaacatcctgctgtcagagccagggttagtgaagctaggggactttggttctgcgtccatcatggcacctgccaactccttcgtgggcaccccatactggatggcacccgaggtgatcctggccatggatgaggggcagtacgatggcaaagtggacgtctggtccttggggataacctgcatcgagctggctgaacggaaaccaccgctctttaacatgaatgcgatgagtgccttataccacattgcacagaacgaatcccccgtgctccagtcaggacactggtctgagtacttccggaattttgtcgactcctgtcttcagaaaatccctcaagacagaccaacctcagaggttctcctgaagcaccgctttgtgctccgggagcggccacccacagtcatcatggacctgatccagaggaccaaggatgccgtgcgggagctggacaacctgcagtaccgcaagatgaagaagatcctgttccaagaggcacccaacggccctggtgccgaggccccagaggaggaagaggaggccgagccctacatgcaccgggccgggactctgaccagcctcgagagtagccactcagtgcccagcatgtccatcagcgcctccagccagagcagctccgtcaacagcctagcagatgcctcagacaacgaggaagaggaggaggaggaggaggaagaggaggaggaggaagaaggccctgaagcccgggagatggccatgatgcaggagggggagcacacagtcacctctcacagctccattatccaccggctgccgggctctgacaacctatatgatgacccctaccagccagagataacccccagccctctccagccgcctgcagccccagctcccacttccaccacctcttctgcccgccgccgggcctactgccgtaaccgagaccactttgccaccatccgaaccgcctccctggtcagccgtcagatccaggagcatgagcaggactctgcgctgcgggagcagctgagcggctataagcggatgcgacgacagcaccagaagcagctgctggccctggagtcacggctgaggggtgaacgggaggagcacagtgcacggctgcagcgggagcttgaggcgcagcgggctggctttggggcagaggcagaaaagctggcccggcggcaccaggccataggtgagaaggaggcacgagctgcccaggccgaggagcggaagttccagcagcacatccttgggcagcagaagaaggagctggctgccctgctggaggcacagaagcggacctacaaacttcgcaaggaacagctgaaggaggagctccaggagaaccccagcactcccaagcgggagaaggccgagtggctgctgcggcagaaggagcagctccagcagtgccaggcggaggaggaagcagggctgctgcggcggcagcgccagtactttgagctgcagtgtcgccagtacaagcgcaagatgttgctggctcggcacagcctggaccaggacctgctgcgggaggacctgaacaagaagcagacccagaaggacttggagtgtgcactgctgcttcggcagcacgaggccacgcgggagctggagctgcggcagctccaggccgtgcagcgcacgcgggctgagctcacccgcctgcagcaccagacggagctgggcaaccagctggagtacaacaagcggcgtgagcaagagttgcggcagaagcatgcggcccaggttcgccagcagcccaagagcctcaaatctaaggagctgcagatcaagaagcagttccaggagacgtgtaagatccagactcggcagtacaaggctctgcgagcacacttgctggagaccacgcccaaagctcagcacaagagcctccttaagcggctcaaggaagagcagacccgcaagctggcgatcttggcggagcagtatgaccagtccatctcagagatgctcagctcacaggcgctgcggcttgatgagacccaggaggcagagttccaggcccttcggcagcagcttcaacaggagctggagctgctcaacgcttaccagagcaagatcaagatccgcacagagagccagcacgagagggagctgcgggagctggagcagagggtcgcgctgcggcgggcactgctggagcagcgggtggaagaggagctgctggccctgcagacaggacgctccgagcgaatccgcagtctgcttgagcggcaggcccgtgagatcgaggccttcgatgcggaaagcatgaggctgggcttctccagcatggctctggggggcatcccggctgaagctgctgcccagggctatcctgctccaccccctgccccagcctggccctcccgtcccgttccccgttctggggcacactggagccatggccctcctccaccaggcatgccccctccagcctggcgtcagccgtctctgctggctcccccaggccccccaaactggctggggccccccacacaaagtgggacaccccgtggcggagccctgctgctgctaagaaacagcccccagcccctgcggcgggcagcctcggggggcagtggcagtgagaatgtgggcccccctgctgccgcggtgcccgggcccctgagccgcagcaccagtgtcgcttcccacatcctcaatggttcttcccacttctattcctgaggtgcagcggggaggagcagatgagctgggcagggcaggggtgggtggagcctgaccctggagggcactgagctggaggcccctgcaagggtaggggacaagatgtaggctccagctcccctcagacctcctcatctcatgagcttcttggggctggccagtggcccagggccagcttggcgatagatgcctcaaggctgcctgggagccccgcctccctaccatggtgccaggggtctccctccgccacctaggaaaggagggagatgtgcgtgtcaaatattcatctagtcccctgggggaggggaagggtgggtctagacatactatattcagagaactatactaccctcacagtgaggccctcagacctgccacagggcagagcaggtctggggcctgaggcagggagaatgagaggccaccttactggcaggaaggatcaggatggggtcttggggtcaggatgcctgggtctcttcccgtaactgtctgacgtcctgtgccgtcttgtcctttatcttttttttttttttttaattgggatcagggctggggcggggaaacaagggaaggaccttggaaggggctgctcccaggcctggggggcagtcgtgggagcccctctcagctgtggggctggcacagagccccaggcaagcttttaataaactgttggttattctaac

SEQ ID NO: 8 - protein, 1049 aa, Homo sapiens
  ref <308>: DATABASE ACCESSION NUMBER: Genbank NP_004774.1
  ref <309>: DATABASE ENTRY DATE: 2000-11-01
  ref <313>: RELEVANT RESIDUES: (1)..(1049)
MPAGGRAGSLKDPDVAELFFKDDPEKLFSDLREIGHGSFGAVYFARDVRNSEVVAIKKMSYSGKQSNEKWQDIIKEVRFLQKLRHPNTIQYRGCYLREHTAWLVMEYCLGSASDLLEVHKKPLQEVEIAAVTHGALQGLAYLHSHNMIHRDVKAGNILLSEPGLVKLGDFGSASIMAPANSFVGTPYWMAPEVILAMDEGQYDGKVDVWSLGITCIELAERKPPLFNMNAMSALYHIAQNESPVLQSGHWSEYFRNFVDSCLQKIPQDRPTSEVLLKHRFVLRERPPTVIMDLIQRTKDAVRELDNLQYRKMKKILFQEAPNGPGAEAPEEEEEAEPYMHRAGTLTSLESSHSVPSMSISASSQSSSVNSLADASDNEEEEEEEEEEEEEEEGPEAREMAMMQEGEHTVTSHSSIIHRLPGSDNLYDDPYQPEITPSPLQPPAAPAPTSTTSSARRRAYCRNRDHFATIRTASLVSRQIQEHEQDSALREQLSGYKRMRRQHQKQLLALESRLRGEREEHSARLQRELEAQRAGFGAEAEKLARRHQAIGEKEARAAQAEERKFQQHILGQQKKELAALLEAQKRTYKLRKEQLKEELQENPSTPKREKAEWLLRQKEQLQQCQAEEEAGLLRRQRQYFELQCRQYKRKMLLARHSLDQDLLREDLNKKQTQKDLECALLLRQHEATRELELRQLQAVQRTRAELTRLQHQTELGNQLEYNKRREQELRQKHAAQVRQQPKSLKSKELQIKKQFQETCKIQTRQYKALRAHLLETTPKAQHKSLLKRLKEEQTRKLAILAEQYDQSISEMLSSQALRLDETQEAEFQALRQQLQQELELLNAYQSKIKIRTESQHERELRELEQRVALRRALLEQRVEEELLALQTGRSERIRSLLERQAREIEAFDAESMRLGFSSMALGGIPAEAAAQGYPAPPPAPAWPSRPVPRSGAHWSHGPPPPGMPPPAWRQPSLLAPPGPPNWLGPPTQSGTPRGGALLLLRNSPQPLRRAASGGSGSENVGPPAAAVPGPLSRSTSVASHILNGSSHFYS

SEQ ID NO: 9 - DNA, 3285 bp, Homo sapiens
  misc_feature (3245)..(3245): a, t, c or g
  misc_feature (3263)..(3263): a, t, c or g
  misc_feature (3278)..(3278): a, t, c or g
  misc_feature (72)..(72): a, t, c or g
  ref <308>: DATABASE ACCESSION NUMBER: EMBL AY049015
  ref <309>: DATABASE ENTRY DATE: 2001-10-15
  ref <313>: RELEVANT RESIDUES: (1)..(3285)
caacggatttcatttcatacagatgaaccaaggatcgggatagcagtataaaattagaatcaagacagctgnctgccaagcaggatgccatcaactaacagagcaggcagcctgaaggaccctgaaattgcagagctcttcttcaaagaagatccagagaagctcttcacagatctcagagaaattggccatggaagctttggagcagtgtattttgcacgagatgtgcgtaccaatgaagtggtggccatcaagaaaatgtcttatagtggaaagcagtctactgagaaatggcaggatattattaaggaagtcaagtttctacaaagaataaaacatcccaacagtatagaatacaaaggctgttatttacgtgaacacacagcatggcttgtaatggaatattgtttaggatctgcttcggatttactagaagttcacaaaaagccattacaagaagtggaaatagcagcaattacacatggtgctcttcagggattagcctacttacattctcatactatgattcatagagatatcaaagcaggaaatatccttctgacagaaccaggccaggtgaaacttgctgactttggctctgcttccatggcatcacctgccaattcctttgtgggaacgccgtattggatggccccagaagtaattttagccatggatgaaggacaatatgatggcaaagtagatgtgtggtctcttggaataacatgtattgaactagcggaaaggaagcctcctttatttaatatgaatgcaatgagtgccttatatcacatagcccaaaatgaatcccctacactacagtctaatgaatggtctgattattttcgcaactttgtagattcttgcctccagaaaatccctcaagatcgacctacatcagaggaacttttaaagcacatatttgttcttcgggagcgccctgaaaccgtgttaatagatctcattcagaggacaaaggatgcagtaagagagctggacaatctgcagtatcgaaagatgaagaaactccttttccaggaggcacataatggaccagcagtagaagcacaggaagaagaagaggaacaagatcatggtgttggccggacaggaacagttaatagtgttggaagtaatcaatccattcccagcatgtccatcagtgccagcagccaaagcagtagtgttaacagtcttccagatgtctcagatgacaagagtgagctagacatgatggagggagaccacacagtgatgtctaacagttctgttatccatttaaaaccagaggaagaaaattacagagaagagggagatcctagaacaagagcatcagatccacaatctccaccccaagtatctcgtcacaaatcacactatcgtaatcgagaacactttgctactatacggacagcatcactggttacgaggcaaatgcaagaacatgagcaggactctgagcttagagaacaaatgtctggctataagcgaatgaggcgacaacatcaaaagcaactgatgactctggaaaacaagctaaaggctgagatggatgaacatcgcctcagattagacaaagatcttgaaactcagcgtaacaattttgctgcagaaatggagaaacttatcaagaaacaccaggctgctatggagaaagaggctaaagtgatgtccaatgaagagaaaaaatttcagcaacatattcaggcccaacagaagaaagaactgaatagttttctcgagtcccagaaaagagagtataaacttcgaaaagagcagcttaaagaggagctaaatgaaaaccagagtacccccaaaaaagaaaaacaggagtggctttcaaagcagaaggagaatatacagcatttccaagcagaagaagaagctaaccttcttcgacgtcaaagacaatacctagagctggaatgccgtcgcttcaagagaagaatgttacttgggcgtcataacttagagcaggaccttgtcagggaggagttaaacaaaagacagactcagaaggacttagagcatgccatgctactccgacagcatgaatctatgcaagaactggagttccgccacctcaacacaattcagaagatgcgctgtgagttgatcagattacagcatcaaactgagctcactaaccagctggaatataataagcgaagagaacgagaactaagacgaaagcatgtcatggaagttcgacaacagcctaagagtttgaagtctaaagaactccaaataaaaaagcagtttcaggatacctgcaaaatccaaaccagacagtacaaagcattaagaaatcacctgctggagactacaccaaagagtgagcacaaagctgttctgaaacggctcaaggaggaacagacccggaaattagctatcttggctgagcagtatgatcacagcattaatgaaatgctctccacacaagccctgcgtttggatgaagcacaggaagcagagtgccaggttttgaagatgcagctgcagcaggaactggagctgttgaatgcgtatcagagcaaaatcaagatgcaagctgaggcacaacatgatcgagagcttcgcgagcttgaacagagggtctccctccggagggcactcttagaacaaaagattgaagaagagatgttggctttgcagaatgagcgcacagaacgaatacgaagcctgttggaacgtcaagccagagagattgaagcttttgactctgaaagcatgagactaggttttagtaatatggtgctttctaatctctcccctgaggcattcagccacagctacccgggagcttctggttggtcacacaaccctactgggggtccaggacctcactggggtcatcccatgggtggcccaccacaagcttggggccatccaatgcaaggtggaccccagccatggggtcacccttcagggccaatgcaaggggtacctcgaggtagcagtatgggagtccgcaatagcccccaagctctgaggcggacagcttctgggggacggacggagcagggcatgagcagaagcacgagtgtcacttcacaaatatccaatgggtcacacatgtcttatacataacttaataattgagagtggcaattccgctggagctgtctgccaaaagaaactgcctacagacatcatcacagcagcctcctcacttgggtactacagtgtggaagctgagtgcatatggtatattttattcatttttgtaaagcgttctgttttgngtttactaattgggatgncatagtacttggctncccgggt

SEQ ID NO: 10 - protein, 1005 aa, Homo sapiens
  ref <308>: DATABASE ACCESSION NUMBER: SpTrEMBL
  ref <309>: DATABASE ENTRY DATE: 2000-10-01
  ref <313>: RELEVANT RESIDUES: (1)..(1005)
LLSRMPSTNRAGSLKDPEIAELFFKEDPEKLFTDLREIGHGSFGAVYFARDVRTNEVVAIKKMSYSGKQSTEKWQDIIKEVKFLQRIKHPNSIEYKGCYLREHTAWLVMEYCLGSASDLLEVHKKPLQEVEIAAITHGALQGLAYLHSHTMIHRDIKAGNILLTEPGQVKLADFGSASMASPANSFVGTPYWMAPEVILAMDEGQYDGKVDVWSLGITCIELAERKPPLFNMNAMSALYHIAQNESPTLQSNEWSDYFRNFVDSCLQKIPQDRPTSEELLKHIFVLRERPETVLIDLIQRTKDAVRELDNLQYRKMKKLLFQEAHNGPAVEAQEEEEEQDHGVGRTGTVNSVGSNQSIPSMSISASSQSSSVNSLPDVSDDKSELDMMEGDHTVMSNSSVIHLKPEEENYREEGDPRTRASDPQSPPQVSRHKSHYRNREHFATIRTASLVTRQMQEHEQDSELREQMSGYKRMRRQHQKQLMTLENKLKAEMDEHRLRLDKDLETQRNNFAAEMEKLIKKHQAAMEKEAKVMSNEEKKFQQHIQAQQKKELNSFLESQKREYKLRKEQLKEELNENQSTPKKEKQEWLSKQKENIQHFQAEEEANLLRRQRQYLELECRRFKRRMLLGRHNLEQDLVREELNKRQTQKDLEHAMLLRQHESMQELEFRHLNTIQKMRCELIRLQHQTELTNQLEYNKRRERELRRKHVMEVRQQPKSLKSKELQIKKQFQDTCKIQTRQYKALRNHLLETTPKSEHKAVLKRLKEEQTRKLAILAEQYDHSINEMLSTQALRLDEAQEAECQVLKMQLQQELELLNAYQSKIKMQAEAQHDRELRELEQRVSLRRALLEQKIEEEMLALQNERTERIRSLLERQAREIEAFDSESMRLGFSNMVLSNLSPEAFSHSYPGASGWSHNPTGGPGPHWGHPMGGPPQAWGHPMQGGPQPWGHPSGPMQGVPRGSSMGVRNSPQALRRTASGGRTEQGMSRSTSVTSQISNGSHMSYT

SEQ ID NO: 11 - DNA, 1576 bp, artificial sequence
  RNAi fragment T17E9.1a (kin-18)
cgaaaaccagcagaagagcgaatgtcagctgaagaatgctttcgacatccattcattcaacggtctcgcccatcagacacaattcaggaactcattcagagaacgaaaaatatggtattagagttggataattttcaatacaaaaagatgagaaaactcatgtatttggatgaaacagaaggaaaagaaggaagtgaaggaaatggagcatctgatgatttagattttcatggaaatgaagctaattcaattggaagaggtagtttttaaaattcaaagtgaaaatattaatatcttggaataatttttataatattgctttaaaccctcagcttttttttgcagactctatcccttagttgttcgttttccatctattctcgttttcagcaggagattctgcgtcatctcgaagtgcttctcttacttctttccgatcaatgcagagtagtggaggagctggtcttttagtgtccaccaatacgacgggtgctatggataatgtgcatggtactgtactgttttttttgttttaggaatggctttattatttcctgcaaagttcaaaaattccatttattttagtttttctctcgaaattcatcgcgcaacattgagaatctttcaaaattttcaggatcctctggatacggtaatggaagtagttcgacgacgagctccgcacgccgccgtcctccaattccttcgcaaatgctctcttctacatcaacgtctggtgttggaactatgccgagtcatggatcagttggagcatcgattacggcgatcgcagtcaatccaacaccgtctccttcagaacctatcccaacatcacaaccaacatcgaaatcagaatcatcttctatactcgaaactgcacacgatgatcctttggacacgtcgatacgtgctccagtgaaagacttgcatatgccgcatcgagcagtcaaggaacgaatagccacgttgcaaaatcacaaattcgcgacgcttcgttcccagagaataatcaatcaggaacaagaagaatatacgaaagagaacaatatgtatgagcaaatgagcaagtacaagcatctacgacaagcacatcacaaagagctccaacaatttgaagaacgatgtgcattagatagagagcaactgcgtgtgaaaatggatcgagaactcgaacaattgacaacgacatactcgaaagaaaagatgagagtgaggtgttcacagaataatgaactagacaaacggaaaaaagatatcgaagatggggagaaaaagatgaaaaagacgaaaaatagtcaaaatcagcagcagatgaaactgtattcagcgcaacaattgaaagaatacaagtataacaaggaggcacagaaaacagtgagaattcacttttatttgatttctgtaaagaaattatacgaaatttagactttataaatttttaaatatgaaagttctggtcactttttcagctgcttctccacttttttcaaagtttattatttagtcttgaataattttttaaaaaatgtcctaaaccaagaattttcagcgattacgaagtctgaacatgcctcga

SEQ ID NO: 12 - DNA, 20 bp, artificial sequence
  primer
cgaaaaccagcagaagagcg

SEQ ID NO: 13 - DNA, 21 bp, artificial sequence
  primer
tcgaggcatgttcagacttcg

(19) United States
(12) Patent Application Publication
(10) Pub. No.: US 2006/0275767 A1
(43) Pub. Date: Dec. 7, 2006

(54) Kinase sequences useful for developing compounds for the prevention and/or treatment of metabolic diseases and nucleotide sequences encoding such kinase sequences

(75) Inventor: Gert Jules Hector De Wilde, Zele (BE)

Correspondence Address: WOLF GREENFIELD & SACKS, PC, FEDERAL RESERVE PLAZA, 600 ATLANTIC AVENUE, BOSTON, MA, 02210-2206, US

(73) Assignee: Devgen NV, Zwijnaarde (BE)

(21) Appl. No.: 10/540,634

(22) PCT Filed: Dec. 19, 2003
(86) PCT No.: PCT/EP03/14674

§ 371 Date: Apr. 19, 2006

Related U.S. Application Data
(60) Provisional application No. 60/436,380, filed on Dec. 23, 2002.

(30) Foreign Application Priority Data
Dec. 23, 2002 (GB) 0230014.3

(51) Int. Cl. C12Q 1/68; C07H 21/04; C12P 21/06; A61K 39/395; C12N 9/12

(52) U.S. Cl. 435/6; 435/69.1; 435/194; 435/320.1; 435/325; 536/23.2; 530/388.26; 424/146.1

(57) ABSTRACT

The present invention relates to nucleotide sequences that encode and may be used to express amino acid sequences that are useful in the identification and development of compounds with activity as pharmaceuticals, in particular of compounds for the prevention and treatment of metabolic diseases such as diabetes and obesity. The invention also relates to the amino acid sequences—such as proteins and polypeptides—that are encoded by, and that may be obtained by suitable expression of, the nucleotide sequences of the invention, particularly the amino acid sequences of JIK, PSK, TAO1 and Q9P2I6. The invention also relates to various uses and modulators of, and methods incorporating, the nucleotide and amino acid sequences of the invention.

The present invention relates to nucleotide sequences that are useful in the pharmaceutical field.

In particular, the invention relates to nucleotide sequences that encode and/or may be used to express amino acid sequences that are useful in the identification and/or development of compounds with (potential) activity as pharmaceuticals, in particular of compounds for the prevention and/or treatment of metabolic diseases such as diabetes and obesity. These nucleotide sequences, which will be further described below, will also be referred to herein as “nucleotide sequences of the invention”.

The invention also relates to the amino acid sequences—such as proteins and/or polypeptides—that are encoded by, and/or that may be obtained by suitable expression of, the nucleotide sequences of the invention. These amino acid sequences, which will be further described below, will also be referred to herein as “amino acid sequences of the invention”.

The invention also relates to the use of the nucleotide sequences of the invention, preferably in the form of a suitable genetic construct as described below, in the transformation of host cells and/or host organisms, for example for the expression of the amino acid sequences of the invention. The invention also relates to host cells and/or host organisms that have been transformed with the nucleotide sequences of the invention and/or that can express the amino acid sequences of the invention.

The invention further relates to methods for the identification and/or development of compounds that can modulate the (biological) activity of the amino acid sequences of the invention, in which the abovementioned nucleotide sequences, amino acid sequences, genetic constructs, host cells and/or host organisms are used. Such methods which will usually be in the form of an assay or screen, as will also be further described below.

The invention also relates to the use of the nucleotide sequences, amino acid sequences, genetic constructs, host cells and/or host organisms of the invention in (methods for) identifying and/or developing compounds that can modulate the (biological) activity of the amino acid sequences of the invention.

Also, the invention relates to compounds that can modulate the (biological) activity of the amino acid sequences of the invention, to compositions that contain such compounds, and to the use of such compounds in the preparation of such compositions.

In particular, the invention relates to such compositions that are in the form of pharmaceutical compositions, and more in particular in the form of pharmaceutical compositions for the prevention or treatment of metabolic diseases such as diabetes or obesity, and also to the use of compounds that can modulate the (biological) activity of the amino acid sequences of the invention in the preparation of such pharmaceutical compositions.

The invention further relates to the use of the nucleotide sequences, amino acid sequences, genetic constructs, host cells and/or host organisms of the invention in (methods for) identifying and/or developing compounds that can be used in the prevention or treatment of metabolic diseases.

Other aspects, embodiments, applications and advantages of the present invention will become clear from the further description below.

The present invention was established from the finding that the amino acid sequences of the invention are involved in metabolic processes (as further described below) and thus can be used as (potential) “target(s)” for in vitro and/or in vivo interaction with chemical compounds and other factors (with the term “target” having its usual meaning in the art, vide for example the definition given in WO 98/06737), and also from the finding that the nucleic acid sequence and amino acid sequences of the invention are involved in metabolic diseases. Consequently, compounds and/or factors that have been identified as interacting with the amino acid sequences of the invention (e.g. by the methods as described hereinbelow) may be useful as active agents in the pharmaceutical field, and in particular for the prevention and treatment of metabolic diseases. All this is supported by the following experimental data/observations:
In an experimental model for fat handling, metabolism and storage using the model organism C. elegans (which model is further described in the Examples), downregulation of the C. elegans gene T17E9.1a by RNA interference strongly reduces the fat storage phenotype in said nematode. As further described below, T17E9.1a is the C. elegans ortholog of STE20 like kinase in mammals.
   Some particularly preferred examples of nucleotide sequences of the invention are:
the nucleotide sequence of SEQ ID NO:1 (T17E9.1a, also known as kin-18), which is a sequence derived from the nematode worm C. elegans; and
the human orthologs of said C.elegans sequence, as may be identified by bioinformatic comparison of the C.elegans sequence with the human genome. Some preferred, but non-limiting orthologues are given in SEQ ID NOS: 3, 5, 7 and 9.

Generally herein, the use of the human nucleotide sequences of SEQ ID NOS: 3, 5, 7 and 9 and/or the use of nucleotide sequence derived therefrom will be preferred, in particular when the invention is used to develop compounds for pharmaceutical use.

In a broader sense, the term “nucleotide sequence of the invention” also comprises:
parts and/or fragments of any of the nucleotide sequence of SEQ ID NO'S: 1, 3, 5, 7 and 9;
(natural and/or synthetic) mutants, variants, alleles, analogs, orthologs (hereinbelow collectively referred to as “mutants”) of any of the nucleotide sequence of SEQ ID NO'S: 1-5, as further described below.
parts and/or fragments of such (natural or synthetic) mutants;
nucleotide fusions of any of the nucleotide sequence of SEQ ID NO'S: 1-5 (or a part or fragment thereof) with at least one further nucleotide sequence;
nucleotide fusions of (natural or synthetic) mutants (or a part or fragment thereof) with at least one further nucleotide sequence;
   in which such mutants, parts, fragments and/or fusions are preferably as further described below.

The invention also comprises different splice variants of the above nucleotide sequences.

Some particularly preferred examples of amino acid sequences of the invention are:
the amino acid sequence of SEQ ID NO:2, which is a sequence derived from the nematode worm C. elegans; and
the human analogs of said C.elegans sequence, as may be identified by bioinformatic comparison of the C.elegans sequence with the human genome. Some preferred, but non-limiting analogs are given in SEQ ID NOS: 4, 6, 8 and 10.

In a broader sense, the term “amino acid sequence of the invention” also comprises:
parts and/or fragments of the amino acid sequence of SEQ ID NOS: 2, 4, 6, 8 and 10.
(natural and/or synthetic) mutants, variants, alleles, analogs, orthologs (hereinbelow collectively referred to as “analogs”) of the amino acid sequence of SEQ ID NOS: 2, 4, 6, 8 and 10;
parts and/or fragments of such analogs;
fusions of the amino acid sequence of SEQ ID NOS: 2, 4, 6, 8 and 10 (or a part or fragment thereof) with at least one further amino acid residue or sequence;
fusions of the amino acid sequence of an analog (or a part or fragment thereof) with at least one further amino acid residue or sequence;
   in which such mutants, parts, fragments and/or fusions are preferably as further described below.

The term “amino acid sequence of the invention” also comprises “immature” forms of the abovementioned amino acid sequences, such as a pre-, pro- or prepro-forms and/or fusions with suitable leader sequences. Also, the amino acid sequences of the invention may have been subjected to post-translational processing and/or be suitably glycosylated, depending upon the host cell or host organism used to express/produce said amino acid sequence; and/or may be otherwise modified (e.g. by chemical techniques known per se in the art).

Generally herein, the use of the human amino acid sequence of SEQ ID NOS: 4, 6, 8 and 10 and/or of amino acid sequences derived therefrom will be preferred, in particular when the invention is used to develop compounds for pharmaceutical use.

From the prior art, the following is known about the nucleotide sequences, amino acid sequences and compounds of the invention:
(1) SEQ ID NOS: 1 and 2. These are the nucleotide sequence and amino acid sequence, respectively, of the C. elegans gene/protein T17E9.1a (also known as “kin-18”) Reference is made to the following entry from the WORMBASE database:

for protein sequence:
http://www.wormbase.org/db/seq/protein?name=WP%3ACE01405:class=Protein

for DNA sequence:
http://www.wormbase.org/db/seg/seguence?name=T17E9.1a:class=Sequence

as well as to Berman et al., Gene 279 (2001) 137-147.
(2) SEQ ID NOS: 3 and 4. These are the nucleotide sequence and amino acid sequence, respectively, of the human gene/protein known as “JNK/SAPK inhibitory kinase” (also known as “JIK”, “DPK” or “KDS”, or STE20-like kinase).

Reference is made to the following entries from the GENBANK database:
NM_{—}016281(mRNA); and
NP_{—}057365.2 (translation).

See also:
GENBANK entry AF135158; Zhang et al., Biochem. Biophys. Res. Commun. 274 (3), 872-879 (2000); MEDLINE 20384190, PUBMED 10924369; and
GENBANK entry AF179867; Tassi et al., J. Biol. Chem. 274 (47), 33287-33295 (1999); MEDLINE 20026851; PUBMED 10559204
(3) SEQ ID NOS: 5 and 6. These are the nucleotide sequence and amino acid sequence, respectively, of the human gene/protein known as “prostate derived STE20-like kinase” (also known as “PSK” or “TAO2”).

Reference is made to the following entries from the GENBANK database:
NM_{—}016151.1 (mRNA)
NP_{—}057235.1 (translation)

See also:
GENBANK entry AF061943; Moore et al, J. Biol. Chem. 275 (6), 4311-4322 (2000); MEDLINE 20127920; PUBMED 10660600;
Chen et al., J. Biol. Chem., Vol. 274, No. 40, pp. 288803-28808 (1999).
(4) SEQ ID NOS: 7 and 8. These are the nucleotide sequence and amino acid sequence, respectively, of the human gene/protein known as “thousand and one amino acid protein kinase” (also known as “TAO1”).

Reference is made to the following entries from the GENBANK database:
NM_{—}004783 (mRNA)
NP_{—}004774.1 (tranlation)

See also:
GENBANK entry AB020688; Nagase et al, DNA Res. 5 (6), 355-364 (1998); MEDLINE 99156230; PUBMED 10048485
(5) SEQ ID NOS: 9 and 10. These are the nucleotide sequence and amino acid sequence, respectively, of the human gene/protein which with designation KIAA1361 (note that although this gene and protein are also known as “thousand and one amino acid protein kinase” or “TAO1”, KIAA1361 is a different gene/protein from SEQ ID NOS 7 and 8).

Reference is made to the following entries from the EMBL and SpTrEMBL databases
AY049015 (EMBL)
Q9P2I6 (SPTREMBL)

See also: Nagase et al., DNA Res. 7:65 (2000) and EMBL entry NR AB037782 (http://www.ebi.ac.uk/cgi-bin/emblfetch?AB037782)

Also, although the inventors do not wish to be limited thereto, it is believed that SEQ ID NOS: 5/6 on the one hand, and SEQ ID NOS: 7 and 8 on the other hand, are transcripts from the same gene/locus.

Thus, in a first aspect, the invention relates to a nucleic acid, preferably in (essentially) isolated form, which nucleic acid encodes and/or can be used to express an amino acid sequence of the invention (as defined herein), and in particular the amino acid sequence of SEQ ID NOS: 2, 4, 6, 8 or 10.

In another aspect, the invention relates to a nucleic acid, preferably in (essentially) isolated form, which nucleic acid comprises a nucleotide sequence of the invention, and in particular the nucleotide sequence of SEQ ID NOS: 1, 3, 5, 7 or 9.

In a yet another aspect, the invention relates to a nucleic acid, preferably in (essentially) isolated form, which nucleic acid essentially consists of a nucleotide sequence of the invention, and in particular a nucleotide sequence SEQ ID NO: 1, 3, 5, 7 or 9.

Collectively, these nucleic acids will also be referred to herein as “nucleic acids of the invention”. Also, where appropriate in the context of the further description of the invention below, the terms “nucleotide sequence of the invention” and “nucleic acid of the invention” may be considered essentially equivalent and essentially interchangeable.

Also, for the purposes of the present invention, a nucleic acid or amino acid sequence is considered to “(in) essentially isolated (form)”—for example, from its native biological source—when it has been separated from at least one other component (and in particular macromolecule) with which it is usually associated, such as another nucleic acid, another protein/polypeptide or another (polymeric) biological component. In particular, a nucleic acid or amino acid sequence is considered “essentially isolated” when it has been purified at least 2-fold, in particular at least 10-fold, more in particular at least 100-fold, and up to 1000-fold or more.

The nucleic acids of the invention may also be in the form of a genetic construct, again as further described below. These constructs will also be referred to herein as “genetic constructs of the invention”. In a preferred embodiment, such a construct will comprise:
a) the nucleotide sequence of the invention; operably connected to:
b) one or more regulatory elements, such as a promoter and optionally a suitable terminator;
   and optionally also:
c) one or more further elements of genetic constructs known per se;
   in which the terms “regulatory element”, “promoter”, “terminator”, “further elements” and “operably connected” have the meanings indicated hereinbelow.

In another aspect, the invention relates to a protein or polypeptide, preferably in (essentially) isolated form, said protein or polypeptide comprising an amino acid sequence of the invention (as defined above), and in particular the amino acid sequence of SEQ ID NOS: 2, 4, 6, 8 or 10.

In a further aspect, the invention relates to a protein or polypeptide, preferably in (essentially) isolated form, said protein or polypeptide essentially consisting of an amino acid sequence of the invention (as defined above), and in particular of the amino acid sequence of SEQ ID NOS: 2, 4, 6, 8 or 10.

In a further aspect, the invention relates to methods for transforming a host cell and/or a host organism with a nucleotide sequence, with a nucleic acid and/or with a genetic construct of the invention. The invention also relates to the use of a nucleotide sequence, of a nucleic acid and/or of a genetic construct of the invention transforming a host cell or a host organism.

In yet another aspect, the invention relates to a host cell or host organism that has been transformed and/or contains with a nucleotide sequence, with a nucleic acid and/or with a genetic construct of the invention. The invention also relates to a host cell and/or host organism that expresses, or (at least) is capable of expressing (e.g. under suitable conditions), an amino acid sequence of the invention. Collectively, such host cells/host organisms will also be referred to herein as “host cells/host organisms of the invention”.

In yet another aspect, the invention relates to methods for producing an amino acid sequence of the invention, in which a nucleotide sequence, nucleic acid, genetic construct, host cell or host organism of the invention is used. Such methods may for instance include expressing a nucleotide sequence of the invention in a suitable host cell or host organism (e.g. upon suitable transformation), and/or maintaining and/or cultivating a host cell or host organism of the invention under suitable conditions, i.e. such that an amino acid sequence of the invention is expressed or obtained. Optionally, these methods may also comprise (one or more steps for) isolating the amino acid sequence thus expressed/produced. The invention also relates to the use of a nucleotide sequence, a nucleic acid, a genetic construct and/or a host cell/host organism of the invention in such a method.

In yet a further aspect, the invention relates to a method for identifying a compound that can modulate the (biological) activity of, and/or that can otherwise interact with, an amino acid sequence of the invention, which method is as further described below. The invention also relates to the use of a nucleotide sequence, a nucleic acid, a genetic construct, an amino acid sequence and/or a host cell/host organism of the invention in such a method.

In yet a further aspect, the invention relates to a method for identifying a compound that can be used in (the preparation of a pharmaceutical composition for) the prevention and/or treatment of metabolic diseases (as further defined below), which method is as further described below. The invention also relates to the use of a nucleotide sequence, a nucleic acid, a genetic construct, an amino acid sequence and/or a host cell/host organism of the invention in such a method.

The invention also relates to compounds that can modulate the (biological activity of), and/or that can otherwise interact with, an amino acid sequence of the invention, either in vitro or preferably (also) in vivo, as further described below. The invention also relates to compositions that contain such compounds, and in particular to pharmaceutical compositions that contains such compounds.

The invention further relates to the use of compounds that can modulate the (biological activity of), and/or that can otherwise interact with, an amino acid sequence of the invention in the preparation of these compositions, and in particular to the use of such compounds in the preparation of a pharmaceutical composition for the prevention and/or treatment of metabolic diseases.

The invention also relates to compounds that can be used in the prevention and/or treatment of metabolic diseases (as further defined below), which compounds have or can be identified and/or developed using the method, nucleic acid sequence, amino acid sequence and/or host cell or host organism of the invention. The invention also relates to compositions that contain such compounds, and in particular to pharmaceutical compositions that contain said compounds.

The invention also relates to the use of such compounds in the preparation of a pharmaceutical composition, and in particular to the use of such compounds in the preparation of a pharmaceutical composition for the prevention or treatment of metabolic diseases.

Unless explicitly specified herein, all terms used in the present description have their usual meaning in the art, for which particular reference is made to the definitions given in W098/06737 and EP 1 085 089.

The nucleotide sequences and amino acid sequences of the invention may generally further be characterized by the presence of a kinase domain (identified using SMART™-analysis), as follows:
SEQ ID NO:2: from amino acid (a.a.) residue 30 to a.a. residue 289
SEQ ID NO:4: from a.a. residue 24 to a.a. residue 277
SEQ ID NO:6: from a.a. residue 28 to a.a. residue 281
SEQ ID NO:8: from a.a. residue 28 to a.a. residue 281
SEQ ID NO: 10: from a.a. residue 32 to a.a. residue 285

On the basis of the above and on the basis of the prior art referred to above, and although the invention is not specifically limited to any specific explanation or mechanism, it is assumed that the nucleotide sequences and/or amino acid sequences have (biological) activity as kinases.

In particular, and although the invention is again not limited to any specific explanation or hypothesis, it is assumed that the amino acid sequences of the invention may be involved in (the modulation of) the JNK/SAPK pathway (vide for example Zhang et al. and Tassi et al., both supra; as well as Yoneda et al., J. Biol. Chem., Vol. 276, No.17, p. 13935-13940 (2001)).

For JNKs and their role in diabetes/obesity, reference is inter alia made to Hirosumi et al., Nature. Vol. 20, November 2002, 333-336.

As is known in the art, biological activity of this kind can be measured using standard assay techniques for kinases, which are well known to the skilled person. Some preferred, but non-limiting examples include:
the JNK/SAPK assay described by Zhang et al., supra, which is particularly suitable for the kinase of SEQ ID NO 4 or a similar kinase;
the kinase assays described by Moore et al., supra, in particular the in vitro kinase assays described on page 4315;

The nucleotide sequence of SEQ ID NO: 1 was identified, and can be derived/isolated from/using the nematode C.elegans; in the manner as further described in Berman et al., or in any other suitable manner known per se.

The nucleotide sequences of SEQ ID NOS: 3, 5, 7 and 9 were identified, and can be derived/isolated from/using human cells; in the manner as further described in the prior art referred to above, or in any other suitable manner known per se.

Also, it is expected that—based upon the disclosure herein—the skilled person will be able to identify, derive and/or isolate natural “mutants” (as mentioned above) of the nucleotide sequence of SEQ ID NOS: 1, 3, 5, 7 and/or 9. For example, such mutants could be derived from (other individuals of) the same species (for example from an individual of a different strain or line, including but not limited to mutant strains or lines); and/or from (individuals of) other species (in which case these mutants will also be referred to herein as “orthologs”). Some examples of species from which such orthologs could be derived include, but are not limited to species of
unicellular and/or micro-organisms such as bacteria, and yeast,
invertebrate multicellular organisms as such as insects and nematodes (for example, agronomically harmful insect or nematode species);
vertebrate multicellular organisms as such as fish, birds, reptiles, amphibians and mammals;

Preferably, a natural ortholog is derived from a mammal such as a mouse, rat, rabbit or dog.

Such natural mutants may be obtained in a manner essentially analogous to the methods described in the prior art referred to above, or alternatively by:
construction of a DNA library from the species of interest in an appropriate expression vector system, followed by direct expression of the mutant sequence;
construction of a DNA library from the species of interest in an appropriate expression vector system, followed by screening of said library with a probe of the invention (as described below) and/or with a(nother) nucleotide sequence of the invention;
isolation of mRNA that encodes the mutant sequence from the species of interest, followed by cDNA synthesis using reverse transcriptase;
   and/or by any other suitable method(s) or technique(s) known per se, for which reference is for instance made to the standard handbooks, such as Sambrook et al, “Molecular Cloning: A Laboratory Manual” (2nd.ed.), Vols. 1-3, Cold Spring Harbor Laboratory Press (1989) and F. Ausubel et al, eds., “Current protocols in molecular biology”, Green Publishing and Wiley Interscience, New York (1987).

It is also expected that—based upon the disclosure herein—the skilled person will be able to provide and/or derive synthetic mutants (as defined hereinabove) of the nucleotide sequences of SEQ ID NOS: 1, 3, 5, 7 and/or 9.

Techniques for generating such synthetic sequences will be clear to the skilled person and may for instance include, but are not limited to, automated DNA synthesis; site-directed mutagenesis; combining two or more parts of one or more naturally occurring sequences, introduction of mutations that lead to the expression of a truncated expression product; introduction of one or more restriction sites (e.g. to create casettes and/or regions that may easily be digested and/or ligated using suitable restriction enzymes), and/or the introduction of mutations by means of a PCR reaction using one or more “mismatched” primers, using for example a sequence of a naturally occurring GPCR as a template. These and other techniques will be clear to the skilled person, and reference is again made to the standard handbooks, such as Sambrook et al. and Ausubel et al., mentioned above.

Preferably, any mutants as described herein will encode amino acid sequences having one or more, and preferably all, of the structural characteristics/conserved features referred to above for the sequences of SEQ ID NO: 2, 4, 6, 8 and/or 10, and in particular will contain a kinase domain.

It is also possible in the invention to use a part or fragment of the nucleotide sequences of SEQ ID NOS 1, 3, 5, 7 or 9; or a part or fragment of a (natural or synthetic) mutant thereof. These may for instance be 5′ and/or 3′ truncated nucleotide sequences, or sequences with an introduced in frame startcodon or stopcodon. Also, two or more such parts or fragments of one or more nucleotide sequences of the invention may be suitably combined (e.g. ligated in frame) to provide a (further) nucleotide sequence of the invention.

Preferably, any such parts or fragments will be such that they comprise at least one continuous stretch of at least 15 nucleotides, preferably at least 30 nucleotides, more preferably at least 60 nucleotides, even more preferably more than 90 nucleotides, of one or more of the nucleotide sequences of SEQ ID NOS: 1, 3, 5, 7 and/or 9.

In particular, any mutants, parts or fragments as described herein may be such that they (at least) encode the active/catalytic site of the corresponding amino acid sequence of the invention and/or a binding domain of the corresponding amino acid sequence of the invention

Any mutants, parts and/or fragments as described herein are preferably (also) such that they are capable of hybridizing with one or more of the nucleotide sequences of SEQ ID NOS 1, 3, 5, 7 and/or 9, i.e. under conditions of “moderate stringency”, and preferably under conditions of “high stringency”. Such conditions will be clear to the skilled person, for example from the standard handbooks, such as Sambrook et al. and Ausubel et al., mentioned above, as well as in EP 0 967 284, EP 1 085 089 or WO 00/55318.

In particular, any mutants, parts and/or fragments as described herein may be such that they are capable of hybridizing with the nucleotide sequence of SEQ ID NO 1, 3, 5, 7 and/or 9 under the “stringent” hybridisation conditions described in WO 00/78972 and WO 98/49185, and/or under the hybridization conditions described in GB 2 357 768-A.

Also, any mutants, parts and/or fragments as described herein will preferably have a degree of “sequence identity”, at the nucleotide level, with one or more of the nucleotide sequences of SEQ ID NOS: 1, 3, 5, 7 and/or 9 of at least 50%, preferably at least 60%, more preferably at least 70%, even more preferably at least 80%, and in particular more than 90%, and up to 95% or more.

For this purpose, the percentage of “sequence identity” between a given nucleotide sequence and the nucleotide sequence of SEQ ID NO: 1, 3, 5, 7 and/or 9 may be calculated by dividing [the number ofnucleotides in the given nucleotide sequence that are identical to the nucleotide at the corresponding position in the nucleotide sequence of the relevant SEQ ID NO] by [the total number of nucleotides in the given nucleotide sequence] and multiplying by [100%], in which each deletion, insertion, substitution or addition of a nucleotide—compared to the sequence of the relevant SEQ ID NO—is considered as a difference at a single nucleotide (position).

Alternatively, the degree of sequence identity may be calculated using a known computer algorithm for sequence alignment such as NCBI Blast v2.0, using standard settings.

Some other techniques, computer algorithms and settings for determining the degree of sequence identity are for example described in EP 0 967 284, EP 1 085 089, WO 00/55318, WO 00/78972, WO 98/49185 and GB 2 357 768-A.

Also, in a preferred aspect, any mutants, parts and/or fragments as described herein will encode proteins/polypeptides having a biological activity that is essentially similar to the biological activity described above for the sequences of SEQ ID NOS: 2, 4, 6, 8 and/or 10, i.e. to a degree of at least 10%, preferably at least 50 % more preferably at least 75%, and up to 90%, as measured by a suitable assay for kinase activity known per se, such as those mentioned above (preferred) or below. In particular, they will have activity as a kinase, as measured using a suitable assay of kinase activity, such as those referred to above, and preferably an activity which is at least 10%, preferably at least 50 % more preferably at least 75%, and up to 90% or more, of the kinase activity of any one of the sequences of SEQ ID NOS: 2, 4, 6, 8 and/or 10.

Preferably, any mutants, parts and/or fragments of the nucleotide sequence of the invention will (also) be such that they encode an amino acid sequence which has a degree of “sequence identity”, at the amino acid level, with one or more of the amino acid sequence of SEQ ID NOS: 2, 4, 6, 8 and/or 10 of at least 50%, preferably at least 60%, more preferably at least 70%, even more preferably at least 80%, and in particular more than 90% and up to 95 % or more, in which the percentage of “sequence identity” is calculated as described below.

Preferably, a nucleotide sequence of the invention will (also) have a length (expressed as total number of nucleotides), which is at least 50%, preferably at least 60%, more preferably at least 70%, even more preferably at least 80%, and in particular more than 90% and up to 95 % or more of the length of one or more of the nucleotide sequence of SEQ ID NOS: 1, 3, 5, 7 and/or 9.

Generally, the nucleotide sequences of the invention, when in the form of a nucleic acid, may be DNA or RNA, and may be single stranded or double stranded. For example, the nucleotide sequences of the invention may be genomic DNA, cDNA or synthetic DNA (such as DNA with a codon usage that has been specifically adapted for expression in the intended host cell or host organism). Thus, the nucleotide sequences of the invention may contain intron sequences, and also generally comprises different splice variants.

It is also within the scope of the invention to use a fusion of a nucleotide sequence of the invention (as described above) with one or more further nucleotide sequence(s), including but not limited to one or more coding sequences, non-coding sequences and/or regulatory sequences. Preferably, in such fusions, the one or more further nucleotide sequences are operably connected (as described below) to the nucleotide sequence of the invention (for example so that, when the further nucleotide sequence is a coding sequence, the nucleotide fusion encodes a protein fusion as described below).

Another embodiment of the invention relates to a nucleic acid probe that is capable of hybridizing with a nucleotide sequence of the invention under conditions of moderate stringency, preferably under conditions of high stringency, and in particular under stringent conditions (all as described above). Such nucleotide probes may for instance be used for detecting and/or isolating a(nother) nucleotide sequence of the invention and/or as a primer for amplifying a nucleotide sequence of the invention; all using techniques known per se, for which reference is again made to the general handbooks such as Sambrook et al. and Ausubel et al. mentioned above.

Generally, such probes can be designed by the skilled person starting from a nucleotide sequence and/or amino acid sequence of the invention—and in particular one or more of the sequences of SEQ ID NOS 1, 3, 5, 7 and/or 9—optionally using a suitable computer algorithm. Also, as will be clear to the skilled person, such probes may be degenerate probes.

In another embodiment, the invention relates to an antisense molecule against a nucleotide sequence of the invention.

Yet another embodiment relates to a double stranded RNA molecule directed against a nucleotide sequence of the invention (one strand of which will usually comprise at least part of a nucleotide sequence of the invention). The invention also relates to genetic constructs that can be used to provide such double stranded RNA molecules (e.g. by suitable expression in a host cell or host organism, or for example in a bacterial strain such as E.coli). For such constructs, reference is made to for example the International Applications PCT/IB01/1068 and WO 00/01846, both by applicant.

The amino acid sequence of SEQ ID NO: 2 was identified, and can be derived/isolated from/using the nematode C.elegans; in the manner as further described in Berman et al., or in any other suitable manner known per se.

The amino acid sequences of SEQ ID NOS: 4, 6, 8 and 10 were identified, and can be derived/isolated from/using human cells; in the manner as further described in the prior art referred to above, or in any other suitable manner known per se.

Generally, the amino acid sequences of SEQ ID NOS: 2, 4, 6, 8 and/or 10 may be isolated from the species mentioned above (i.e. C. elegans and human, respectively), using any technique(s) for protein isolation and/or purification known per se. Alternatively, the amino acid sequences of SEQ ID NOS: 2, 4, 6, 8 and/or 10 may be obtained by suitable expression of a suitable nucleotide sequence—such as one of the nucleotide sequences of SEQ ID NOS: 1, 3, 5, 7 and/or 9, as applicable or a suitable mutant thereof—in an appropriate host cell or host organism, as further described below.

Also, it is expected that—based upon the disclosure herein—the skilled person will be able to identify, derive and/or isolate natural “analogs” (as mentioned above) of the amino acid sequences of SEQ ID NOS: 2, 4, 6, 8 and/or 10. Such mutants could be derived from (other individuals of) the same species (for example from an individual of a different strain or line, including but not limited to mutant strains or lines); and/or from (individuals of) other species. For example, such analogs could be derived from the insect species or other pest species mentioned above.

Such natural analogs may again be obtained by isolating them from their natural source using any technique(s) for protein isolation and/or purification known per se, or alternatively by suitable expression of a suitable nucleotide sequence of the invention—such as a natural mutant as described above—in an appropriate host cell or host organism, as further described below.

It is also expected that—based upon the disclosure herein—the skilled person will be able to provide and/or derive synthetic “analogs” (as mentioned above) of one or more of the amino sequences of SEQ ID NOS: 2, 4, 6, 8 and/or 10.

Generally, such synthetic analogs may be obtained by suitable expression of a suitable nucleotide sequence of the invention—such as a synthetic mutant as described above—in an appropriate host cell or host organism, as further described below.

Preferably, any analogs as described herein will have one or more, and preferably all, of the structural characteristics/conserved features referred to above for the sequences of SEQ ID NO: 2, 4, 6, 8 and/or 10, and in particular will contain a kinase domain.

It is also possible in the invention to use a part or fragment of one or more of the amino acid sequences of SEQ ID NOS 2, 4, 6, 8 and/or 10, or a part or fragment of a (natural or synthetic) analog thereof mutant thereof. This may for instance be N- and/or C-truncated amino acid sequence. Also, two or more parts or fragments of one or more amino acid sequences of the invention may be suitably combined to provide a (further) amino acid sequence of the invention.

Preferably, any such parts or fragments will be such that they comprise at least one continuous stretch of at least 5 amino acids, preferably at least 10 amino acids, more preferably at least 20 amino acids, even more preferably more than 30 amino acids, of one or more of the amino acid sequences of SEQ ID NO: 2, 4, 6, 8 and/or 10.

In particular, any parts or fragments as described herein are such that they (at least) comprise the active/catalytic site of the corresponding amino acid sequence of the invention and/or a binding domain of the corresponding amino acid sequence of the invention, and in particular a kinase domain. As will be clear to the skilled person, such parts or fragments may find particular use in assay- and screening techniques (as generally described below) and/or (when said part or fragment is provided in crystalline form) in X-ray crystallography.

Generally, such parts or fragments of the amino acid sequences of the invention may be obtained by suitable expression of a suitable nucleotide sequence of the invention—such as a suitable part or fragment as described hereinabove for the nucleotide sequences of the invention—in an appropriate host cell or host organism, as further described below.

In addition and/or as an alternative to the methodology above, amino acid sequences of the invention may also be provided by (chemically and/or enzymatically) modifying the side chain(s) of one or more amino acid residues of an amino acid sequence of SEQ ID NO: 2, 4, 6, 8 and/or 10 or a part, fragment, (natural and/or synthetic) mutant, variant, allele, analogs, orthologs thereof, for example by one or more of the side chain modifications as described in WO 01/02560 and/or by incorporating (e.g. by insertion and/or substitution) one or more unnatural amino acid residues, again as described in WO 01/02560.

Preferably, any analogs, parts and/or fragments as described herein will be such that they have a degree of “sequence identity”, at the amino acid level, with one or more of the amino acid sequences of SEQ ID NOS 2, 4, 6, 8 and/or 10 of at least 50%, preferably at least 60%, more preferably at least 70%, even more preferably at least 80%, and in particular more than 90% and up to 95 % or more.

For this purpose, the percentage of “sequence identity” between a given amino acid sequence and one of the amino acid sequences of SEQ ID NOS: 2, 4, 6, 8 and/or 10 may be calculated by dividing [the number of amino acid residues in the given amino acid sequence that are identical to the amino acid residue at the corresponding position in the amino acid sequence of the relevant SEQ ID NO] by [the total number of amino acid residues in the given amino acid sequence] and multiplying by [100%], in which each deletion, insertion, substitution or addition of an amino acid residue—compared to the sequence of the relevant SEQ ID NO—is considered as a difference at a single amino acid (position).

Alternatively, the degree of sequence identity may be calculated using a known computer algorithm, such as those mentioned above.

Also, such sequence identity at the amino acid level may take into account so-called “conservative amino acid substitutions”, which are well known in the art, for example from GB-A-2 357 768, WO 98/49185, WO 00/46383 and WO 01/09300; and (preferred) types and/or combinations of such substitutions may be selected on the basis of the pertinent teachings from the references mentioned in WO 98/49185.

Also, preferably, any analogs, parts and/or fragments as described herein will have a biological activity that is essentially similar to the biological activity described above for the sequences of SEQ ID NOS: 2, 4, 6, 8 and/or 10, i.e. to a degree of at least 10%, preferably at least 50 %, more preferably at least 75%, and up to 90%, as measured by the assay mentioned above. In particular, they will have activity as a kinase, as measured using a suitable assay of kinase activity, such as those referred to above, and preferably an activity which is at least 10%, preferably at least 50 %, more preferably at least 75%, and up to 90% or more, of the kinase activity of any one of the sequences of SEQ ID NOS: 2, 4, 6, 8 and/or 10.

Preferably, an amino acid sequence of the invention will (also) have a length (expressed as total number of amino acid residues), which is at least 50%, preferably at least 60%, more preferably at least 70%, even more preferably at least 80%, and in particular more than 90% and up to 95 % or more of the length of one or more of the amino acid sequence of SEQ ID NOS: 2, 4, 6, 8 and/or 10.

It is also within the scope of the invention to use a fusion of an amino acid sequence of the invention (as described above) with one or more further amino acid sequences, for example to provide a protein fusion. Generally, such fusions may be obtained by suitable expression of a suitable nucleotide sequence of the invention—such as a suitable fusion of a nucleotide sequence of the invention with one or more further coding sequences—in an appropriate host cell or host organism, as further described below. According to one non-limiting embodiment, such a fusion comprises at least the kinase domain of one of the amino acid sequences of the invention.

In one particular embodiment, such fusions may comprise an amino acid sequence of the invention (that preferably at least comprises the kinase domain, and may essentially consist of the kinase domain) fused with a reporter protein such as GFP, luciferase or another fluorescent protein moiety. As will be clear to the skilled person, such fusions may find particular use in expression analysis and similar methodologies.

In another embodiment, the fusion partner may be an amino acid sequence or residue that may be used in purification of the expressed amino acid sequence, for example using affinity techniques directed against said sequence or residue. Thereafter, said sequence or residue may be removed (e.g. by chemical or enzymatical cleavage) to provide the nucleotide sequence of the invention (for this purpose, the sequence or residue may optionally be linked to the amino acid sequence of the invention via a cleavable linker sequence). Some preferred, but non-limiting examples of such residues are multiple histidine residues and glutatione residues,

In one preferred, but non-limiting aspect, any such fusion will have a biological activity that is essentially similar to the biological activity described above for the sequences of SEQ ID NOS: 2, 4, 6, 8 and/or 10, i.e. to a degree of at least 10%, preferably at least 50 %, more preferably at least 75%, and up to 90%, as measured by the assay mentioned above. In particular, they will have activity as a kinase, as measured using a suitable assay of kinase activity, such as those referred to above, and preferably an activity which is at least 10%, preferably at least 50 %, more preferably at least 75%, and up to 90% or more, of the kinase activity of any one of the sequences of SEQ ID NOS: 2, 4, 6, 8 and/or 10.

Genetic constructs of the invention will generally comprise at least one nucleotide sequence of the invention, optionally linked to one or more elements of genetic constructs known per se, as described below.

Such genetic constructs may be DNA or RNA, and are preferably double-stranded DNA. The constructs may also be in a form suitable for transformation of the intended host cell or host organism, in a form suitable for integration into the genomic DNA of the intended host cell or in a form suitable for independent replication, maintenance and/or inheritance in the intended host organism. For instance, the genetic construct may be in the form of a vector, such as for example a plasmid, cosmid, YAC, a viral vector or transposon. In particular, the vector may be an expression vector, i.e. a vector that can provide for expression in vitro and/or in vivo (e.g. in a suitable host cell and/or host organism as described below).

As the one or more “further elements” referred to above, the genetic construct(s) of the invention may generally contain one or more suitable regulatory elements (such as a suitable promoter(s), enhancer(s), terminator(s), etc.), 3′- or 5′-UTR sequences, leader sequences, selection markers, expression markers/reporter genes, and/or elements that may facilitate or increase (the efficiency of) transformation or integration. These and other suitable elements for such genetic constructs will be clear to the skilled person, and may for instance depend upon the type of construct used, the intended host cell or host organism; the manner in which the nucleotide sequences of the invention of interest are to be expressed (e.g. via constitutive, transient or inducible expression); and/or the transformation technique to be used.

Preferably, in the genetic constructs of the invention, the one or more further elements are “operably linked” to the nucleotide sequence(s) of the invention and/or to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promotor)

Generally, when two nucleotide sequences are operably linked, they will be in the same orientation and usually also in the same reading frame. They will usually also be essentially contiguous, although this may also not be required.

Preferably, the optional further elements of the genetic construct(s) used in the invention are such that they are capable of providing their intended biological function in the intended host cell or host organism.

For instance, a promoter, enhancer or terminator should be “operable” in the intended host cell or host organism, by which is meant that (for example) said promoter should be capable of initiating or otherwise controlling/regulating the transcription and/or the expression of a nucleotide sequence—e.g. a coding sequence—to which it is operably linked (as defined above).

Such a promoter may be a constitutive promoter or an inducible promoter, and may also be such that it (only) provides for expression in a specific stage of development of the host cell or host organism, and/or such that it (only) provides for expression in a specific cell, tissue, organ or part of a multicellular host organism.

Some particularly preferred promoters include, but are not limited to those present in the expression vectors referred to below.

A selection marker should be such that it allows—i.e. under appropriate selection conditions—host cells and/or host organisms that have been (successfully) transformed with the nucleotide sequence of the invention to be distinguished from host cells/organisms that have not been (successfully) transformed. Some preferred, but non-limiting examples of such markers are genes that provide resistance against antibiotics (such as kanamycine or ampicilline), genes that provide for temperature resistance, or genes that allow the host cell or host organism to be maintained in the absence of certain factors, compounds and/or (food) components in the medium that are essential for survival of the non-transformed cells or organisms.

A leader sequence should be such that—in the intended host cell or host organism—it allows for the desired post-translational modifications and/or such that it directs the transcribed mRNA to a desired part or organelle of a cell. A leader sequence may also allow for secretion of the expression product from said cell. As such, the leader sequence may be any pro-, pre-, or prepro-sequence operable in the host cell or host organism.

An expression marker or reporter gene should be such that—in the host cell or host organism—it allows for detection of the expression of (a gene or nucleotide sequence present on) the genetic construct. An expression marker may optionally also allow for the localisation of the expressed product, e.g. in a specific part or organelle of a cell and/or in (a) specific cell(s), tissue(s), organ(s) or part(s) of a multicellular organism. Such reporter genes may also be expressed as a protein fusion with the amino acid sequence of the invention. Some preferred, but non-limiting examples include fluorescent proteins such as Green Fluorescent Protein (GFP).

For some (further) non-limiting examples of the promoters, selection markers, leader sequences, expression markers and further elements that may be present/used in the genetic constructs of the invention—such as terminators, transcriptional and/or translational enhancers and/or integration factors—reference is made to the general handbooks such as Sambrook et al. and Ausubel et al. mentioned above, to W. B. Wood et al., “The nematode Caenorhabditis elegans”, Cold Spring Harbor Laboratory Press (1988) and D. L. Riddle et al., “C. ELEGANS II”, Cold Spring Harbor Laboratory Press (1997), as well as to the examples that are given in WO 95/07463, WO 96/23810, WO 95/07463, WO 95/21191, WO 97/11094, WO 97/42320, WO 98/06737, WO 98/21355, U.S. Pat. No. 6,207,410, U.S. Pat. No. 5,693,492 and EP 1 085 089. Other examples will be clear to the skilled person.

The genetic constructs of the invention may generally be provided by suitably linking the nucleotide sequence(s) of the invention to the one or more further elements described above, for example using the techniques described in the general handbooks such as Sambrook et al. and Ausubel et al., mentioned above.

Often, the genetic constructs of the invention will be obtained by inserting a nucleotide sequence of the invention in a suitable (expression) vector known per se. Some preferred, but non-limiting examples of suitable expression vectors include:
vectors for expression in mammalian cells: pMAMneo (Clontech), pcDNA3 (Invitrogen), pMC1neo (Stratagene), pSG5 (Stratagene), EBO-pSV2-neo (ATCC 37593), pBPV-1 (8-2) (ATCC 37110), pdBPV-MMTneo (342-12) (ATCC 37224), pRSVgpt (ATCC37199), pRSVneo (ATCC37198), pSV2-dhfr (ATCC 37146), pUCTag (ATCC 37460) and 1ZD35 (ATCC 37565);
vectors for expression in bacterials cells: pET vectors (Novagen) and pQE vectors (Qiagen);
vectors for expression in yeast or other fungal cells: pYES2 (Invitrogen) and Pichia expression vectors (Invitrogen);
vectors for expression in insect cells: pBlueBacII (Invitrogen).

The nucleotide sequences and/or genetic constructs of the invention may be used to transform a host cell or host organism.

The host cell may be any suitable (fungal, prokaryotic or eukaryotic) cell or cell line, for example:
a bacterial strain, including but not limited to strains of E.coli, Bacillus., Streptomyces and Pseudomonas;
a fungal cell, including but not limited to cells from species of Aspergillus and Trichoderma;
a yeast cell, including but not limited to cells from species of Kluyveromyces or Saccharomyces;
an amphibian cell or cell line, such as Xenopus oocytes.

In one specific embodiment, which may prove particularly useful when the nucleotide sequences of the invention are (to be) used in the discovery and development of insecticidal compounds, the host cell may be an insect-derived cell or cell line, such as:
cells/cell lines derived from lepidoptera, including but not limited to Spodoptera SF9 and Sf21 cells,
cells/cell lines derived from Drosophila, such as Schneider and Kc cells; and/or
cells/cell lines derived from a pest species of interest (as mentioned below), such as from Heliothis virescens.

In one preferred embodiment, the host cell is a mammalian cell or cell line, for example derived from the mammals referred to above.

In an even more preferred aspect, the host cell is a cell or cell line derived from a human, and from other mammals including but not limited to CHO- and BHK-cells and human cells or cell lines such as HeLa and COS.

In one specific, but non-limiting embodiment, the cell or cell line may be a human cell or cell line which is related to metabolic processes or metabolic disease and/or used as a cellular model for metabolic disease, including but not limited to liver cells or cell lines, adipocytes or muscle cells or cell lines such as HEPG2 cells, 3T3L1 adipocytes, CTC12 cells and L6 myotubes.

The host organism may be any suitable multicellular (vertebrate or invertebrate) organism, including but not limited to:
a nematode, including but not limited to nematodes from the genus Caenorhabditis, such as C.elegans,
an insect, including but not limited to species of Drosophila and/or a specific pest species of interest (such as those mentioned above);
other well known model organisms, such as zebrafish;
a mammal such as a rat or mouse;

Other suitable host cells or host organisms will be clear to the skilled person, for example from the handbooks and patent applications mentioned above.

It should be noted that when a nucleotide sequence of the invention is expressed in a multicellular organism, it may be expressed throughout the entire organism, or only in one or more specific cells, tissues, organs and/or parts thereof, for example by expression under the control of a promoter that is specific for said cell(s), tissue(s), organ(s) or part(s).

The nucleotide sequence may also be expressed during only a specific stage of development or life cycle of the host cell or host organism, again for example by expression under the control of a promoter that is specific for said stage of development or life cycle. Also, as already mentioned above, said expression may be constitutive, transient and/or inducible.

According to one specific embodiment, the expression of a nucleotide sequence of the invention in a host cell or host organism may be party or totally reduced (i.e. knocked out), compared to the original (e.g. native) host cell or host organism. This may for instance be achieved in a transient manner using antisense and/or RNA-interference techniques well known in the art, or in a constitutive manner using random, site specific and/or chemical mutagenesis of the nucleotide sequence of the invention, or any other suitable techniques for generating “knock-down” or “knock-out” animals.

Suitable transformation techniques will be clear to the skilled person and may depend on the intended host cell/host organism and the genetic construct to be used. Some preferred, but non-limiting examples of suitable techniques include ballistic transformation, (micro-)injection, transfection (e.g. using suitable transposons), electroporation and lipofection. For these and other suitable techniques, reference is again made to the handbooks and patent applications mentioned above.

After transformation, a step for detecting and selecting those host cells or host organisms that have been succesfully transformed with the nucleotide sequence/genetic construct of the invention may be performed. This may for instance be a selection step based on a selectable marker present in the genetic construct of the invention or a step involving the detection of the amino acid sequence of the invention, e.g. using specific antibodies.

The transformed host cell (which may be in the form or a stable cell line) or host organisms (which may be in the form of a stable mutant line or strain) form further aspects of the present invention.

Preferably, these host cells or host organisms are such that they express, or are (at least) capable of expressing (e.g. under suitable conditions), an amino acid sequence of the invention (and in case of a host organism: in at least one cell, part, tissue or organ thereof). The invention also includes further generations, progeny and/or offspring of the host cell or host organism of the invention, that may for instance be obtained by cell division or by sexual or asexual reproduction.

To produce/obtain expression of the amino acid sequences of the invention, the transformed host cell or transformed host organism may generally be kept, maintained and/or cultured under conditions such that the (desired) amino acid sequence of the invention is expressed/produced. Suitable conditions will be clear to the skilled person and will usually depend upon the host cell/host organism used, as well as on the regulatory elements that control the expression of the (relevant) nucleotide sequence of the invention. Again, reference is made to the handbooks and patent applications mentioned above in the paragraphs on the genetic constructs of the invention.

Generally, suitable conditions may include the use of a suitable medium, the presence of a suitable source of food and/or suitable nutrients, the use of a suitable temperature, and optionally the presence of a suitable inducing factor or compound (e.g. when the nucleotide sequences of the invention are under the control of an inducible promoter); all of which may be selected by the skilled person. Again, under such conditions, the amino acid sequences of the invention may be expressed in a constitutive manner, in a transient manner, or only when suitably induced.

It will also be clear to the skilled person that the amino acid sequence of the invention may (first) be generated in an immature form (as mentioned above), which may then be subjected to post-translational modification, depending on the host cell/host organism used. Also, the amino acid sequence of the invention may be glycosylated, again depending on the host cell/host organism used.

The amino acid sequence of the invention may then be isolated from the host cell/host organism and/or from the medium in which said host cell or host organism was cultivated, using protein isolation and/or purification techniques known per se, such as (preparative) chromatography and/or electrophoresis techniques, differential precipitation techniques, affinity techniques (e.g. using a specific, cleavable amino acid sequence fused with the amino acid sequence of the invention) and/or preparative immunological techniques (i.e. using antibodies against the amino acid sequence to be isolated).

In one embodiment, the amino acid sequence thus obtained may also be used to generate antibodies specifically against said sequence or an antigenic part or epitope thereof.

Such antibodies, which form a further aspect of the invention, may be generated in a manner known per se, for example as described in GB-A-2 357 768, U.S. Pat. No. 5,693,492, WO 95/32734, WO 96/23882, WO 98/02456, WO 98/41633 and/or WO 98/49306, and/or as described in the prior art referred to above. Often, but not exclusively, such methods will involve immunizing an immunocompetent host with the pertinent amino acid sequence of the invention or an immungenic part thereof (such as a specific epitope), in amount(s) and according to a regimen such that antibodies against said amino acid sequence are raised, and than harvesting the antibodies thus generated, e.g. from blood or serum derived from said host.

For instance, polyclonal antibodies can be obtained by immunizing a suitable host such as a goat, rabbit, sheep, rat, pig or mouse with (an epitope of) an amino acid sequence of the invention, optionally with the use of an immunogenic carrier (such as bovine serum albumin or keyhole limpet hemocyanin) and/or an adjuvant such as Freund's, saponin, ISCOM's, aluminium hydroxide or a similar mineral gel, or keyhole limpet hemocyanin or a similar surface active substance. After a suitable immune response has been raised (usually within 1-7 days), the antibodies can be isolated from blood or serum taken from the immunized animal in a manner known per se, which optionally may involve a step of screening for an antibody with desired properties (i.e. specificity) using known immunoassay techniques, for which reference is again made to for instance WO 96/23882.

Monoclonal antibodies may, for example, be produced using continuous cell lines in culture, including hybridoma-based and similar techniques, again essentially as described in the above cited references. Accordingly, cells and cell lines that produce monoclonal antibodies against an amino acid sequence of the invention form a further aspect of the invention, as do methods for producing antibodies against amino acid sequences of the invention, which methods may generally involve cultivating such a cell and isolating the antibodies from the culture (medium), again using techniques known per se.

Also, Fab-fragments against the amino acid sequences of the invention (such as F(ab)₂, Fab′ and Fab fragments) may be obtained by digestion of an antibody with pepsin or another protease, reducing disulfide-linkages and treatment with papain and a reducing agent, respectively. Fab-expression libraries may, for instance, be obtained by the method of Huse et al., 1989, Science 245:1275-1281.

In another embodiment, the nucleotide sequences of the invention, the amino acid sequences of the invention, and/or a host cell or host organism that expresses such an amino acid sequence, may also be used in an assay or assay method generally (including but not limited to diagnostic assays and/or assays to determing the presence and/or absence of specific mutations and/or genetic markers, for example to determine susceptibility for a condition or disease associated with such a mutation or marker), and in particular in an assay to identify and/or (further) develop compounds and/or other factors that can modulate the (biological) activity of, and/or that can otherwise interact with, the amino acid sequences of the invention, and such uses form further aspects of the invention. As will be clear to the skilled person, in this context, the amino acid sequence of the invention will serve as a target for interaction with such a compound or factor

In this context, the terms “modulate”, “modulation, “modulator” and “target” will have their usual meaning in the art, for which reference is inter alia made to the definitions given in WO 98/06737. Generally, a modulator is a compound or factor that can enhance, inhibit/reduce or otherwise alter, influence or affect (collectively referred to as “modulation”) a functional property of a biological activity or process (for example, the biological activity of an amino acid sequence of the invention).

In this context, the amino acid sequence of the invention may serve as a target for modulation in vitro (e.g. as part of an assay or screen) and/or for modulation in vivo (e.g. for modulation by a compound or factor that is known to modulate the target, which compound or factor may for example be used as an active compound for agrochemical, veterinary and/or pharmaceutical use).

For example, the amino acid sequences, host cells and/or host organisms of the invention may be used as part of an assay or screen that may be used to identify and/or develop modulators of the amino acid sequence of the invention, such as a primary screen (e.g. a screen used to identify modulators of the target from a set or library of test chemicals with unknown activity with respect to the target) and/or a secondary assay (e.g. an assay used for validating hits from a primary screen and/or used in optimizing hit molecules, e.g. as part of hits-to-leads chemistry).

For instance, such an assay or screen may be configured as an in vitro assay or screen, which will generally involve binding of the compound or factor to be tested as a potential modulator for the target (hereinbelow also referred to as “test chemical”) to the target, upon which a signal generated by said binding is measured. Suitable techniques for such in vitro screening will be clear to the skilled person, and are for example described in Eldefrawi et al., (1987). FASEB J., Vol.1, pages 262-271 and Rauh et al., (1990), Trends in Pharmacol. Sci., vol.11, pages 325-329. For example, such an assay or screen may be configured as a binding assay or screen, in which the test chemical is used to displace a detectable ligand from the target (e.g. a radioactive or fluorescent ligand), upon which the amount of ligand displaced from the target by the modulator is determined.

Assays aimed at identification of small molecule inhibitors of protein kinases are well known. There is a large number of technologies that can be classified in two main approaches:
1. Detection based on an antibody specifically recognizing the phosphorylated product (peptide) of the kinase action

These approaches rely on the specificity of antibody to differentiate between the peptide containing a phosporylated amino acid vs. the non-phosporylated peptide. This recognition can utilize, for example, the following readouts:
fluorescence polarization (FP), where the detection is based on the change of the overall size upon antibody binding to fluorescent labeled peptide.
fluorescence intensity (FI), where the antibody binding causes change in the intensity of the emission from a fluorescently labeled peptide.
time-resolve fluorescence (where both the antibody and the peptide are labeled with different fluorescent labels and the binding leads to energy transfer between these two labels. This in turn leads to shortening of the donor label fluorescence lifetime. These assays have at least two different embodiments: biochemical or cell-based, via enzyme complementation (the latter is marketed as HitHunter™, by Discoverex).
2. Detection based on other reagents specifically recognizing phosphate groups. Recently, at least three new techniques have been developed that do not rely on the use of specific antibody.
   Trivalent metal ion containing beads. These bind specifically to phosphate groups. The detection is than performed by change in fluorescence polarization, the same as above, upon antibody binding. (IMAP™, Molecular Devices))
   based on chemical modification of the phosphate group and a specific label in the substrate to yield a change in fluorescence intensity. (IQ™, Pierce)
   based on chemical modification of the phosphate group to yield a biotin derivative. The detection is performed via FP upon avidin or strepavidin binding. (Caliper)”

Such an assay or screen may also be configured as a cell-based assay or screen, in which a host cell of the invention is contacted with/exposed to a test chemical, upon which at least one biological response by the host cell is measured.

Suitable cells or cell lines for such cell based assays include those mentioned above. In one preferred, but non-limiting embodiment, the cell or cell line may be a mammalian, and in particular human, cell or cell line which is related to metabolic processes or metabolic disease and/or used as a cellular model for metabolic disease, including but not limited to liver cells or cell lines, adipocytes or muscle cells or cell lines such as HEPG2 cells, 3T3L1 adipocytes, CTC12 cells and L6 myotubes.

Also, such an assay or screen may also be configured as a whole animal screen, in which a host organism of the invention is contacted with/exposed to a test chemical, upon which at least one biological response (such as a phenotypical, behavioural and/orphysiological change, including but not limited to paralysis or death) by the host organism is measured. Such screens may be carried out in any model organism known per se, including but not limited to yeast, Drosophila, zebrafish or C. elegans.

Thus, generally, the assays and screens described above will comprise at least one step in which the test chemical is contacted with the target (and/or with a host cell or host organism that expresses the target), and in particular in such a way that a signal is generated that is representative for the modulation of the target by the test chemical (including modulation of the interaction of the target protein with any other proteins. Suitable techniques for screening such protein-protein interactions, such as FRET, SPA and BRET, will be clear to the skilled person). In a further step, said signal may then be detected.

Accordingly, in one aspect, the invention relates to a method for generating a signal that is representative for the interaction of an amino acid sequence of the invention with a test chemical, said method at least comprising the steps of:
a) contacting the amino acid sequence of the invention, or a host cell or host organism containing/expressing an amino acid sequence of the invention, with said test chemical, in such a way that a signal may be generated that is representative for the interaction between said test chemical and said amino acid sequence; and optionally
b) detecting the signal that may thus be generated.

In another aspect, the invention relates to a method for identifying modulators of an amino acid sequence of the invention (e.g. from a set or library of test chemicals), said method at least comprising the steps of:
a) contacting the amino acid sequence of the invention, or a host cell or host organism containing/expressing an amino acid sequence of the invention, with a test chemical, in such a way that a signal may be generated that is representative for the interaction between said test chemical and said amino acid sequence; and optionally
b) detecting the signal that may thus be generated, said signal identifying a modulator of said amino acid sequence.

Compounds that may be tested using the methods of the invention are generally described below.

The assays and screens of the invention may be carried out at medium throughput to high throughput, for example in an automated fashion using suitable robotics. In particular, in this embodiment, the method of the invention may be carried out by contacting the target with the test compound in a well of a multi-well plate, such as a standard 24, 96, 384, 1536 or 3456 well plate.

Usually, in a screen or assay of the invention, for each measurement, the target or host cell/host organism will be contacted with only a single test compound. However, it is also within the scope of the invention to contact the target with two or more test compounds—either simultaneously or sequentially—for example to determine whether said combination provides a synergistic effect.

Once a test chemical has been identified as a modulator for an amino acid sequence of the invention (e.g. by means of a screen or assay as described hereinabove), it may be used per se as a modulator of the amino relevant amino acid sequence of the invention (e.g. as an active substance for pharmaceutical use), or it may optionally be further optimized for final use, e.g. to improve properties such as solubility, ADME-TOX (see for example http://www.scherago.com/admet/ or http://www.lifesciencesinfo.com/admet) and other desired properties. It will be clear to the skilled person that the nucleotide sequences, amino acid sequences, host cells/host organisms and/or methods of the invention may find further use in such optimization methodology, for example as (part of) secondary assays.

The invention is not particularly limited to any specific manner or mechanism in/via which the modulator (e.g. the test chemical, compound and/or factor) modulates, or interacts with, the target (in vivo and/or in vitro). For example, the modulator may comprise an agonist, an antagonist, an inverse agonist, a partial agonist, a competitive inhibitor, a non-competitive inhibitor, a cofactor, an allosteric inhibitor or other allosteric factor for the target, and/or may be a compound or factor that enhances or reduces binding of target to another biological component associated with its (biological) activity, such as another protein or polypeptide, a receptor, or a part or organelle of a cell. As such, the modulator may bind with the target (at the active site, at an allosteric site, at a binding domain and/or at another site on the target, e.g. covalently or via hydrogen bonding), block the active site of the target (in a reversible, irreversible or competitive manner), block a binding domain of the target (in a reversible, irreversible or competitive manner), and/or influence or change the conformation of the target.

As such, the test chemical/modulator may for instance be:
an analog of a known substrate of the target;
an oligopeptide, e.g. comprising between 2 and 20, preferably between 3 and 15 amino acid residues;
an antisense or double stranded RNA molecule;
a protein, polypeptide;
a cofactor or an analog of a cofactor.

Preferably, the compound is an inhibitor of the target, although the invention in its broadest sense is not limited thereto.

The test chemical/modulator may also be a reference compound or factor, which may be a compound that is known to modulate or otherwise interact with the target (e.g. a known substrate or inhibitor for the target) or a compound or factor that is generally known to modulate or otherwise interact with other members from the general class to which the target belongs (e.g. a known substrate or inhibitor of said class).

Preferably, however, the compound(s) will be “small molecules”, by which is generally meant herein a molecular entity with a molecular weight of less than 1500, preferably less than 1000. This may for example be an organic, inorganic or organometallic molecule, which may also be in the form or a suitable salt, such as a water-soluble salt; and may also be a complex, chelate and/or a similar molecular entity, as long as its (overall) molecular weight is within the range indicated above.

In a preferred embodiment, such a “small molecule” has been designed according, and/or meets the criteria of, at least one, preferably at least any two, more preferably at least any three, and up to all of the so-called Lipinski rules for drug likeness prediction (vide Lipinksi et al., Advanced Drug Delivery Reviews 23 (1997), pages 3-25). As is known in the art, small molecules which meet these criteria are particularly suited (as starting points) for the design and/or development of pharmaceuticals for human use, and may for instance be used as starting points for hits-to-leads chemistry, and/or as starting points for lead development (in which the methods of the invention may also be applied).

Also, for these purposes, the design of such small molecules (as well as the design of libraries consisting of such small molecules) will preferably also take into account the presence of pharmacophore points, for example according to the methods described by I. Muegge et al., J. Med. Chem. 44, 12 (2001), pages 1-6 and the documents cited herein.

The term “small peptide” generally covers (oligo)peptides that contain a total of between 2 and 35, such as for example between 3 and 25, amino acids (e.g. in one or more connected chains, and preferably a single chain). It will be clear that some of these small peptides will also be included in the term small molecule as used herein, depending on their molecular weight.

In one preferred, but non-limiting embodiment, the invention is used to screen a set or library of (related or otherwise unrelated) small molecules, for example a standard “robustness set”, a primary screening library (e.g. of otherwise unrelated compounds), a combinatorial library, a series of closely related chemical analogos. Such sets or libraries will be clear to the skilled person, and may for instance include, but are not limited to, such commercially available chemical libraries such as the various libraries available from Tocris Cookson, Bristol, UK.

In yet a further aspect, the invention relates to a method for identifying a compound that can be used in (the preparation of a pharmaceutical composition for) the prevention and/or treatment of metabolic diseases (e.g. from a set or library of test chemicals), said method at least comprising the steps of:
a) contacting an amino acid sequence of the invention, and/or a host cell or host organism containing/expressing an amino acid sequence of the invention, with a test chemical, in such a way that a signal may be generated that is representative for the interaction between said test chemical and said amino acid sequence of the invention; and optionally
b) detecting the signal that may thus be generated, said signal identifying a modulator of said amino acid sequence.

The modulators thus identified can be used in (the preparation of a pharmaceutical composition for) the prevention and/or treatment of metabolic diseases, and/or can be used to develop other compounds that can be used in (the preparation of a pharmaceutical composition for) the prevention and/or treatment of metabolic diseases, i.e. as already outlined above.

The invention also relates to the use of an amino acid sequence and/or a host cell/host organism of the invention in such a method.

Also, as already mentioned above, the use of the human nucleotide sequences of SEQ ID NOS: 3, 5, 7 and/or 9 and/or of sequences derived therefrom (such as mutants, parts, fragments and/or fusions thereof as described hereinabove), of the human amino acid sequences of SEQ ID NOS: 4, 6, 8 and/or 10 and/or of sequences derived therefrom (such as analogs, parts, fragments, and/or fusions thereof as described hereinabove), and of host cells/host organisms containing/expressing these, are usually preferred, in particular when the invention is used to develop compounds for pharmaceutical use.

As already mentioned above, the compounds and/or factors that have been identified and/or developed as modulators of the amino acid sequences of the invention (and/or precursors for such compounds) may be useful as active substances in the pharmaceutical field, for example in the preparation of pharmaceutical compositions, and both such modulators as well as (pharmaceutical) compositions containing them comprise further aspects of the invention.

In particular, the compounds and composition of the invention may be used in (the preparation of pharmaceutical compositions for) the prevention (e.g. prophylaxis) and/or treatment of metabolic diseases (which for the purposes herein in its broadest sense also includes preventing, treating and/or alleviating the symptoms and/or complications of such metabolic diseases).

In particular, such compounds and composition may be used in (the preparation of pharmaceutical compositions for) the prevention (e.g. prophylaxis) and/or treatment of metabolic diseases (which for the purposes herein in its broadest sense also includes preventing, treating and/or alleviating the symptoms and/or complications of such metabolic diseases).

In particular, the compounds and compositions of the invention may be used for preventing and/or treating:
hyperglycemic conditions and/or other conditions and/or diseases that are (primarily) associated with (the response or sensitivity to) insulin, including but not limited to all forms of diabetes and disorders resulting from insulin resistance, such as Type I and Type II diabetes, as well as severe insulin resistance, hyperinsulinemia, and hyperlipidemia, e.g., obese subjects, and insulin-resistant diabetes, such as Mendenhall's Syndrome, Werner Syndrome, leprechaunism, lipoatrophic diabetes, and other lipoatrophies;
conditions caused or usually associated with hyperglycemic conditions and/or obesity, such as hypertension, osteoporosis and/or lipodystrophy.
so-called “metabolic syndrome” (also known as “Syndrome X”) which is a condition where several of the following conditions coexist: hypertension; insulin resistance; diabetes; dyslipidemia; and/or obesity.

In particular, the compounds and compositions of the invention may be used for preventing and/or treating diabetes, especially Type I and Type II diabetes. “Diabetes” itself refers to a progressive disease of carbohydrate metabolism involving inadequate production or utilization of insulin and is characterized by hyperglycemia and glycosuria.

Also, as mentioned above, the amino acid sequences of the invention and in particular the nucleotide sequences of the invention, and more in particular the human amino acid sequences and nucleotide sequences of the invention may be used for diagnostic purposes, for example as part of diagnostic assays and/or as part of kits for performing such assays (in which such a kit will comprise at least a nucleotide sequence of the invention) may be suitably packaged (e.g. in a suitbale container) and may optionally further comprise one or more elements for such kits known per se, such as suitable reagents, buffers or other solvents, and instructions for use).

In particular, the amino acid sequences and nucleotide sequences of the invention, as well as assays and kits using such sequences, may be used for diagnostic purposes relating to one or more of the metabolic diseases specified, for example as assays to determine the presence and/or absence in an individual of specific mutations and/or genetic markers that relate to one or more of the metabolic diseases specified above, to determine the susceptibility and/or any predisposition to any of said metabolic diseases in an individual, to determine if any genetically determined factors contribute or even cause (in full or in part) said metabolic disease in an individual, determine and/or to confirm the kind of metabolic disease from which an individual suffers, and/or to predict the further progress of such a metabolic disease in an individual. It will also be clear that any results obtained using such a diagnostic method or assay may also provide guidance to the clinician as to how such a metabolic disease should be treated in an individual, e.g. which medication should be prescribed and/or the doage regimen to be used.

It should also be noted that, for the treatment of metabolic disease in humans, the compound used will usually and preferably be an inhibitor of an amino acid sequence of the invention, although the invention is its broadest sense is not limited thereto.

In one specific, but non-limiting, embodiment of the invention, a compound is considered an inhibitor of one of the amino acid sequences of the invention if, in a relevant assay such as the kinase activity assays referred to above (or a suitable modification thereof, for example using partially or fully purified protein), said compound reduces the activity of said amino acid sequence, i.e. by at least 1%, preferably at least 10%, such as by 20% or more, compared to the activity without the presence of said compound.

In an even more specific, but non-limiting, embodiment of the invention, a compound is considered an inhibitor of one of the amino acid sequences of the invention if, in a relevant assay, such as a binding assay, said compound has an IC50 value of less than 1000 μM, preferably at than 500 μM, more preferably less than 250 μM, even more preferably less than 100 μM, for example 50 μM or less, such as about 10 μM or less.

Again, preferably, in the invention compounds are used that are modulators, and in particular inhibitors, of the human amino acid sequences of SEQ ID NO: 4, 6, 8 and/or 10, and/or of amino acid sequences derived therefrom, such as analogs, mutants, parts, fragments and/or fusions as described above.

For pharmaceutical use, the compounds of the invention may be used as a free acid or base, and/or in the form of a pharmaceutically acceptable acid-addition and/or base-addition salt (e.g. obtained with non-toxic organic or inorganic acid or base), in the form of a hydrate, solvate and/or complex, and/or in the form or a pre-drug, such as an ester. Such salts, hydrates, solvates, etc. and the preparation thereof will be clear to the skilled person; reference is for instance made to the salts, hydrates, solvates, etc. described in U.S. Pat. No. 6,372,778, U.S. Pat. No. 6,369,086 and U.S. Pat. No. 6,369,067

Generally, for pharmaceutical use, the compounds of the inventions may be formulated as a pharmaceutical preparation comprising at least one compound of the invention and at least one pharmaceutically acceptable carrier, diluent or excipient and/or adjuvant, and optionally one or more further pharmaceutically active compounds. By means of non-limiting examples, such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such suitable administration forms—which may be solid, semi-solid or liquid, depending on the manner of administration—as well as methods and carriers for use in the preparation thereof, will be clear to the skilled person; reference is again made to for instance U.S. Pat. No. 6,372,778, U.S. Pat. No. 3,696, 086 and U.S. Pat. No. 6,369,067.

The pharmaceutical preparations of the invention are preferably in a unit dosage form, and may be suitably packaged, for example in a box, blister, vial, bottle, sachet, ampoule or in any other suitable holder or container (which may be properly labeled); optionally with one or more leaflets containing product information and/or instructions for use. Generally, such unit dosages will contain between I and 500 mg of the at least one compound of the invention, e.g. about 10, 25, 50, 100, 200, 500 or 1000 mg per unit dosage.

For pharmaceutical use, at least one compound of the invention will generally be administered in an amount of between 0.0 1 to 150 mg/kg body weight per day of the patient, divided over one or more daily doses. The amount(s) to be administered and the further treatment regimen may be determined by the treating clinician, depending on factors such as the age, gender and general condition of the patient and the nature and severity of the disease/symptoms to be treated.

Thus, in a further aspect, the invention relates to a composition, and in particular a composition for pharmaceutical use, that contains at least one compound of the invention (i.e. a compound that has been identified, discovered and/or developed using a nematode or method as described herein) and at least one suitable carrier (i.e. a carrier suitable for pharmaceutical use). The invention also relates to the use of a compound of the invention in the preparation of such a composition.

Preferably, the compounds and compositions of the invention are administered orally and/or in a form intended and/or suitable for oral administration.

It is also envisaged that the above compounds and compositions may be of value in the veterinary field, which for the purposes herein not only includes the prevention and/or treatment of diseases in animals, but also—for economically important animals such as cattle, pigs, sheep, chicken, fish, etc.—enhancing the growth and/or weight of the animal and/or the amount and/or the quality of the meat or other products obtained from the animal. Thus, in a further aspect, the invention relates to a composition for veterinary use that contains at least one compound of the invention (i.e. a compound that has been identified, discovered and/or developed using a nematode or method as described herein) and at least one suitable carrier (i.e. a carrier suitable for veterinary use). The invention also relates to the use of a compound of the invention in the preparation of such a composition.

In the agrochemical field, the invention may be used to identify compounds suitable for use in pesticides, insecticides, nematicides and/or other biocides or plant protection agents. For example, the compounds of the invention may be used to control the species listed in U.S. Pat. No. 6,372,774. For this purpose, the compounds of the invention (or a suitable salt, hydrate or ester thereof) may be suitably formulated with one or more agrochemically acceptable carriers, to provide a formulation suitable for agrochemical use, as will be clear to the skilled person (reference is, for example, made to the formulations and uses described in U.S. Pat. No. 6,372,774).

Thus, in a further aspect, the invention relates to a composition for agrochemical use that contains at least one compound of the invention (i.e. a compound that has been identified, discovered and/or developed using a nematode or method as described herein) and at least one suitable carrier (i.e. a carrier suitable for agrochemical use). The invention also relates to the use of a compound of the invention in the preparation of such a composition.

The invention will now be further illustrated by means of the following non-limiting Experimental Part.

In the Figures:

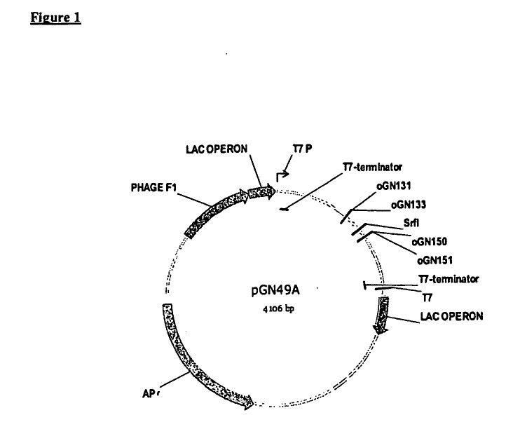

FIG. 1 schematically shows vector pGN49A (see also also WO 00/01846 and British patent application 0012233, both by Applicant);

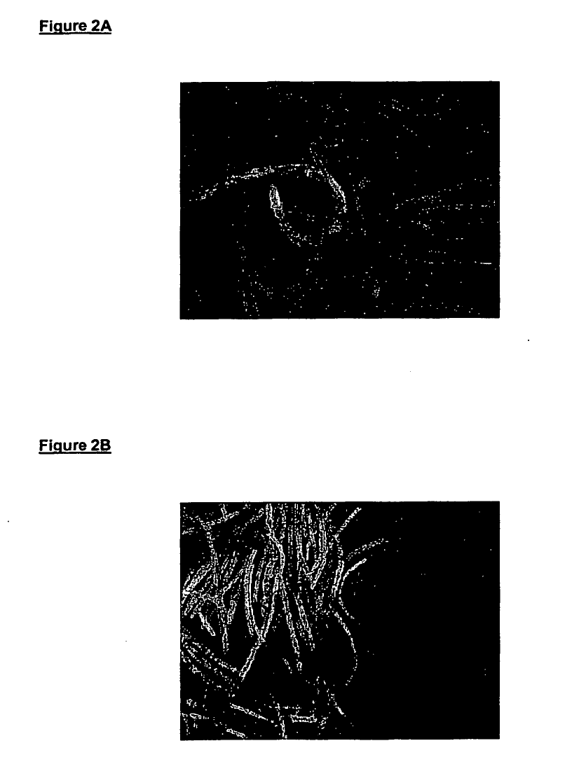

FIGS. 2A and 2B are photographs (enhanced using the Scion Image (Scion Corp) software package) showing reduced fat-absorption phenotype in C. elegans upon Nile Red Staining: FIG. 2A=reduced fat storage (T17E9.1a—invention); FIG. 2B=reference (vector gGN29 without T17E9.1a).

### EXPERIMENTAL PART

In the Experimental Part below, unless indicated otherwise, all steps for handling and cultivating C. elegans were performed using standard techniques and procedures, for which reference is made to the standard C. elegans handbooks, such as W. B. Wood et al., “The nematode Caenorhabditis elegans”, Cold Spring Harbor Laboratory Press (1988); D. L. Riddle et al., “C. ELEGANS II”, Cold Spring Harbor Laboratory Press (1997); “Caenorhabditis elegans, Modern Biological analysis of an organism”: ed. by H. Epstein and D. Shakes, Methods in Cell Biology, Vol 48, 1995; and “C. elegans, a practical approach”, ed. by I. A. Hope, Oxford University Press Inc. New York, USA, 1999.

Downregulation of the gene(s) of interest in C. elegans was achieved by RNAi feeding techniques using an E.coli strain capable of expressing a dsRNA corresponding to the gene(s) of interest, as generally described in—inter alia—the International application WO 00/01846 by applicant and the handbooks referred to above.

Also, unless indicated otherwise, all cloning and other molecular biology steps were performed using standard techniques and protocols, i.e. as provided by the manufacturers of the reagents/kits used and/or as described in the standard handbooks, such as Sambrook et al, “Molecular Cloning: A Laboratory Manual” (2nd.ed.), Vols. 1-3, Cold Spring Harbor Laboratory Press (1989) and F. Ausubel et al, eds., “Current protocols in molecular biology”, Green Publishing and Wiley Interscience, New York (1987).

Fat accumulation in C. elegans daf-2 (e1370) was determined visually under a microscope upon staining with Nile-red, using an adaptation of the general methodology described by Ogg et al., Nature, Vol. 389, 994 (1997). For the general methodology, reference is also made to Thaden et al., 1999 International Worm Meeting abstract 837; Ashrafi and Ruvkun, 2000 East Coast Worn Meeting abstract 67; Ashrafi, Chang and Ruvkun, 2001 International Worrn Meeting abstract 325; and Rottiers and Antebi, 2001 International Worm Meeting abstract 620 (all abstracts available from Worrn Literature Index at http://elegans.swmed.edu/wli/).

### EXAMPLE 1

### Preparation E.coli RNA Feeding Strain for Expression of T17E9.1a Double Stranded RNA

A vector for expression of dsRNA for downregulation of C. elegans gene T17E9.1a (kin-18) was prepared as follows.

The DNA fragment of SEQ ID NO: 11, which corresponds to 1576 nucleotides (26% compared to the genomic sequence) of the C. elegans T17E9.1a gene, was obtained by PCR from genomic C. elegans DNA, using the following primers:

[SEQ ID NO:12]

| | |
|---|---|
| forward primer: | CGAAAACCAGCAGAAGAGCG |

[SEQ ID NO:13]

| | |
|---|---|
| reverse primer: | TCGAGGCATGTTCAGACTTCG |

This fragment was inserted in the SrfI-site of expression vector pGN49a pGN49A (FIG. 1, see also WO 00/01846 and British patent application 0012233, both by Applicant). This vector contains two T7 promoters flanking the SrfI-site, allowing transcription of a nucleotide sequence inserted into said SrfI-site into double stranded RNA, upon binding of a T7 polymerase to said promoter (vide WO 00/01846).

The resulting vector, designated pGN49A-T17E9.1a, was transformed overnight into E. coli strain AB 309-105 (see EP-A-1 093 526 by applicant, page 17.).

To normalize the culture, 250 μl of the overnight culture (1 ml) was transferred to a 96 well plate and the OD at 600 nm was measured (Fluostar Galaxy plate reader BMG), the remaining 750 μl centrifuged down. Next the pellet was re-suspended in S-complete fed ( S-complete supplemented with 0.1 mg/ml ampiciline and 1 mM IPTG) and volume adjusted to obtain OD₆₀₀ value of 1

### EXAMPLE 2

### Generation of Fat Storage Phenotype in C. elegans

In this example, C. elegans strain CB1370 containing the temperature sensitive daf-2 allele e-1370 is used (Ogg et al., supra). CB 1370 is publicly available from, for example, the Caenorhabditis Genetics Center (CGC), Minnesota, USA).

To generate the fat-storage phenotype, L1 worms of strain CB 1370 were cultivated at a temperature of 15° C. in S-Complete fed-medium in the wells of a 96 well plate (1 L1 nematodes per well) under essentially synchronized conditions, until the nematodes reached the L2 stage.

Then, the temperature was increased to 20° C., and the worms were further cultivated at said temperature until their F1 offspring reached the L4 stage (about 144 hrs). Due to the presence of the daf-2 allele e-1370, this raise in temperature from 15° C. to 20° C. causes the nematodes to accumulate fat, mainly in their intestinal and hypodermal tissue (vide Ogg et al. and FIGS. 2A and 2B).

The accumulation of fat (in the form of droplets) was made visible by means of Nile Red staining: L4 animals were washed several times with M9 (supplemented with 0.1% PEG) to remove the remaining E.coli, and fixed with MeOH (fc. 33%). After fixation the nematodes were stained with nile red (fc 0.375 mM in 37.5% MeOH) for 4 hours. MeOH and excess dye was removed through several washes with M9 (supplemented with 0.1% PEG). The staining pattern was visualized under UV using a 500 nm long pass filter.

For testing the influence of the gene T17E9.1a on fat storage, during the steps described above, the worms were grown on 30 μl of the normalized E.coli pGN49A-T17E9.1a strain of Example 1 (OD₆₀₀=1) as a food source. As a reference, the daf-2 (e1370) nematodes were grown in a similar manner, but with E. coli strain AB 309-105 containing vector pGN29A without the T17E9.1a insert as a food source, used in the same amount. All samples were carried out in quadruplicate.

The results were as follows: worms fed on E.coli pGN49A-T17E9.1a strain, which downregulates the expression of T17E9.1a through RNA interference, showed a strong reduction of the accumulation of fat, compared to the reference (vide FIGS. 2A and 2B).

These results show that T17E9.1a is involved in the regulation of the (daf-2 dependent) daf-pathway and the accumulation of fat in the nematode. It is known in the art that both are models for insulin resistance and fat handling in mammals, such as humans.

Bio-informatic analysis of the sequence of T17E9.1a (kin-18) showed that the amino acid sequence of the kin-18 protein (SEQ ID NO:2) has the following degree of sequence identity (in percentages at the amino acid level) with the human amino acid sequences of SEQ ID NOS: 4, 6, 8 and 10, respectively:
SEQ ID NO:4 (“JIK”): overall sequence: 36%; kinase domain: 61%
SEQ ID NO:6 (“PSK”): overall sequence: 24%; kinase domain: 59%
SEQ ID NO:8 (“TAO1”): overall sequence: 32%; kinase domain: 59%
SEQ ID NO:10 (“Q9P216”): overall sequence: 33%; kinase domain: 61%

### EXAMPLE 3

### Screening of SEO ID NO. 4 (“JIK”)

A protein with the amino acid sequence of SEQ ID NO: 4, isolated and purified in a manner known per se, were used to screen a reference set of 96 commercially available kinase inhibitors (obtained from several commercial sources such as Acros Organics, Belgium/USA; Alexis Biochemicals, UK; Biomol, USA; Calbiochem, USA; ICN Biomedicals; Sigma; and Tocris, UK), using the commercially available Kinase Glo® Luminescent Kinase Assay (Promega, USA), essentially according to the instructions of the manufacturer.

In this assay, 6 out of 96 compounds showed a percentage which differed more than 3 Standard Deviation (SD) from 0 (zero) inhibition. These 6 compounds were designated as hits, corresponding with a hit-rate of about 6%.

### EXAMPLE 4

### Screening of SEO ID NO. 8 (“TAO1”)

A protein with the amino acid sequence of SEQ ID NO: 8, isolated and purified in a manner known per se, were used to screen a reference set of 96 commercially available kinase inhibitors (obtained from several commercial sources such as Acros Organics, Belgium/USA; Alexis Biochemicals, UK; Biomol, USA; Calbiochem, USA; ICN Biomedicals; Sigma; and Tocris, UK), using the commercially available Kinase Glo® Luminescent Kinase Assay (Promega, USA), essentially according to the instructions of the manufacturer.

In this assay, 6 out of 96 compounds showed a percentage which differed more than 3 Standard Deviation (SD) from 0 (zero) inhibition. These 6 compounds were designated as hits, corresponding with a hit-rate of about 6%.

1.-17. (canceled)

18. A method for identifying a compound useful in the prevention and/or treatment of metabolic diseases, comprising the steps of:
a) contacting a protein or polypeptide encoded by a T17E9.1a/kin-18 gene or an ortholog thereof with a test chemical, in such a way that a signal is generated that is representative for the interaction between said test chemical and said protein or polypeptide; and
b) detecting the signal thus generated, said signal identifying a compound that modulates said protein or polypeptide, which is indicative that the compound is useful for the prevention and/or treatment of metabolic diseases.

19. The method of claim 18, wherein the ortholog is a human JNK/SAPK inhibitory kinase (JIK) gene, a prostate derived STE20-like kinase (PSK or TAO2) gene, or a thousand and one amino acid protein kinase gene (TAO1 and Q9P2I6).

20. The method of claim 18, wherein the T17E9.1 a/kin-18 gene or ortholog thereof encodes a protein or polypeptide comprising one of the amino acid sequences of SEQ ID NO: 2, SEQ ID NO: 4, SEQ ID NO: 6, SEQ ID NO: 8 and SEQ ID NO: 10; or an analog, variant, allele or ortholog that has 95% or more sequence identity with SEQ ID NO: 2, SEQ ID NO: 4, SEQ ID NO: 6, SEQ ID NO: 8 or SEQ ID NO: 10; or a part, fragment or truncated version comprising a kinase domain thereof.

21. The method of claim 18, wherein the T17E9.1a/kin-18 gene or ortholog thereof is a nucleic acid comprising the nucleotide sequence of SEQ ID NO: 1, SEQ ID NO: 3, SEQ ID NO: 5, SEQ ID NO: 7 or SEQ ID NO: 9; or a mutant, variant, allele, analog or ortholog that has 95% or more sequence identity with SEQ ID NO: 1, SEQ ID NO: 3, SEQ ID NO: 5, SEQ ID NO: 7 or SEQ ID NO: 9; or a part, fragment or truncated version encoding a kinase domain thereof.

22. The method of claim 18, further comprising comparing the signal generated to a signal generated by a control assay.

23. The method of claim 20, wherein the kinase domain comprises amino acid residues 30 to 289 of SEQ ID NO:2, amino acid residues 24 to 277 of SEQ ID NO:4, amino acid residues 28 to 281 of SEQ ID NO:6, amino acid residues 28 to 281 of SEQ ID NO:8, or amino acid residues 32 to 285 of SEQ ID NO:10.

24. A method for identifying a compound useful in the prevention and/or treatment of metabolic diseases, comprising the steps of:
a) contacting host cells or organisms transformed with and/or containing a T17E9.1a/kin-18 nucleic acid sequence or an ortholog thereof, which nucleic acid sequence or ortholog expresses and/or produces a T17E9.1a/kin-18 or orthologous amino acid sequence, with a test chemical, in such a way that a signal is generated that is representative for the interaction between said test chemical and said protein or polypeptide; and
b) detecting the signal thus generated, said signal identifying a compound that modulates said protein or polypeptide, which is indicative that the compound is useful for the prevention and/or treatment of metabolic diseases.

25. The method of claim 24, wherein the ortholog is a human JNK/SAPK inhibitory kinase (JIK) gene, a prostate derived STE20-like kinase (PSK or TAO2) gene, or a thousand and one amino acid protein kinase gene (TAO1 and Q9P2I6).

26. The method of claim 24, wherein the T17E9.1a/kin-18 or orthologous amino acid sequence comprises SEQ ID NO: 2, SEQ ID NO: 4, SEQ ID NO: 6, SEQ ID NO: 8 or SEQ ID NO: 10; or an analog, variant, allele or ortholog that has 95% or more sequence identity with SEQ ID NO: 2, SEQ ID NO: 4, SEQ ID NO: 6, SEQ ID NO: 8 or SEQ ID NO: 10; or a part, fragment or truncated version comprising a kinase domain thereof.

27. The method of claim 24, wherein the T17E9.1a/kin-18 gene or ortholog thereof is a nucleic acid comprising the nucleotide sequence of SEQ ID NO: 1, SEQ ID NO: 3, SEQ ID NO: 5, SEQ ID NO: 7 or SEQ ID NO: 9; or a mutant, variant, allele, analog or ortholog that has 95% or more sequence identity with SEQ ID NO: 1, SEQ ID NO: 3, SEQ ID NO: 5, SEQ ID NO: 7 or SEQ ID NO: 9; or a part, fragment or truncated version encoding a kinase domain thereof.

28. The method of claim 24, further comprising comparing the signal generated to a signal generated by a control assay.

29. The method of claim 26, wherein the kinase domain comprises amino acid residues 30 to 289 of SEQ ID NO:2, amino acid residues 24 to 277 of SEQ ID NO:4, amino acid residues 28 to 281 of SEQ ID NO:6, amino acid residues 28 to 281 of SEQ ID NO:8, or amino acid residues 32 to 285 of SEQ ID NO: 10.
